(19) United States
(12) Patent Application Publication
(10) Pub. No.: US 2021/0290557 A1
(43) Pub. Date: Sep. 23, 2021

(54) MULTI-PARTICULATE PHARMACEUTICAL COMPOSITION, IMMEDIATE RELEASE PELLETS, SUSTAINED RELEASE PELLETS, ENTERIC RELEASE PELLETS AND USE THEREOF

(71) Applicant: Industrial Technology Research Institute, Hsinchu (TW)

(72) Inventors: Yu-Feng HUANG, Taichung City (TW); Chu-Hsun LU, Kaohsiung City (TW); Kuo-Kuei HUANG, Zhubei City (TW); I-Hong PAN, Zhubei City (TW)

(73) Assignee: Industrial Technology Research Institute, Hsinchu (TW)

(21) Appl. No.: 17/126,901

(22) Filed: Dec. 18, 2020

Related U.S. Application Data
(60) Provisional application No. 62/951,427, filed on Dec. 20, 2019.

(51) Int. Cl. A61K 9/50; A61K 9/00; A61K 9/48; A61K 31/047; A61K 31/235; A61K 31/216; A61K 31/075; A61K 31/122; A61K 31/343

(52) U.S. Cl. CPC A61K 9/5084; A61K 9/0002; A61K 9/4808; A61K 31/047; A61K 31/343; A61K 31/216; A61K 31/075; A61K 31/122; A61K 31/235

(57) ABSTRACT

A multi-particulate pharmaceutical composition includes mixture pellets including at least two of the following pellets: a first immediate-release pellet, a sustained-release pellet, an enteric-release pellet and an enteric coated sustained-release pellet.

### CROSS REFERENCE TO RELATED APPLICATION

This application claims the benefit of U.S. Provisional Application No. 62/951,427, filed on Dec. 20, 2019, the entirety of which is incorporated by reference herein.

### TECHNICAL FIELD

The present disclosure is related to drug release particles and applications thereof, and in particular it is related to a multi-particulate pharmaceutical composition, an immediate-release pellet, a sustained-release pellet, an enteric-release pellet, an enteric coated sustained-release pellet, and the use thereof.

### BACKGROUND

Although there are currently multiple treatment options for autoimmune neurological diseases and/or neurodegenerative diseases, such as multiple sclerosis (MS), there is no method for curing said conditions.

Multiple sclerosis (MS) is a disease that causes demyelination of spinal nerve and brain cells and is considered an autoimmune neurological disease. Patients with multiple sclerosis may experience a wide range of symptoms. Because of the nature of multiple sclerosis, symptoms can vary widely from person to person. Moreover, due to the possibility of multiple symptoms at the same time, patients with such diseases may need to use multiple different drugs at the same time.

However, since the frequency and dosage of various drugs are different, the simultaneous use of multiple drugs may cause problems for patients.

Therefore, there is an urgent need for a novel drug delivery system for autoimmune neurological diseases and/or neurodegenerative diseases, which has more than one drug release rate and/or frequency, thereby effectively reducing the number of drug administrations and maintaining the required effects of the drugs within a certain period of time.

### SUMMARY

The present disclosure provides multi-particulate pharmaceutical composition, comprising: mixture pellets, which comprise at least two of the following pellets: a first immediate-release pellet, a first sustained-release pellet, an enteric-release pellet and an enteric coated sustained-release pellet. The first immediate-release pellet is composed of a first medicated mixture, and the first medicated mixture comprises: a first compound, or its geometric isomer, enantiomer, diastereomer or pharmaceutically acceptable salt; and a first excipient composition, wherein a content of the first compound in the first medicated mixture is 5-85 wt %, and the first compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 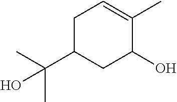 |
| Formula (II) | 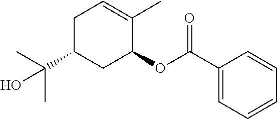 [SMILES: CC1=CC[C@@H](C(C)(C)O)C[C@@H]1OC(=O)c1ccccc1] |
| Formula (III) | 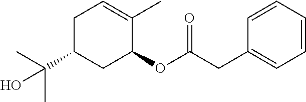 [SMILES: CC1=CC[C@@H](C(C)(C)O)C[C@@H]1OC(=O)Cc1ccccc1] |
| Formula (IV) | 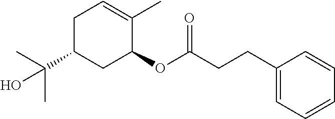 [SMILES: CC1=CC[C@@H](C(C)(C)O)C[C@@H]1OC(=O)CCc1ccccc1] |
| Formula (V) | 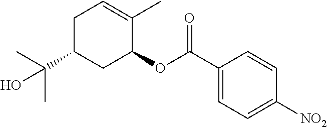 [SMILES: CC1=CC[C@@H](C(C)(C)O)C[C@@H]1OC(=O)c1ccc([N+](=O)[O-])cc1] |
| Formula (VI) | 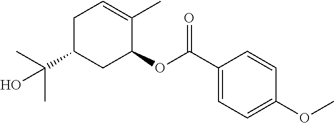 [SMILES: COc1ccc(C(=O)O[C@H]2C[C@H](C(C)(C)O)CC=C2C)cc1] |
| Formula (VII) | 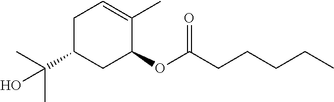 [SMILES: CCCCCC(=O)O[C@H]1C[C@H](C(C)(C)O)CC=C1C] |
| Formula (VIII) | 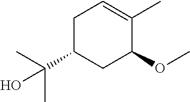 |
| Formula (IX) | 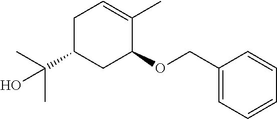 [SMILES: CC1=CC[C@@H](C(C)(C)O)C[C@@H]1OCc1ccccc1] |
| Formula (X) | 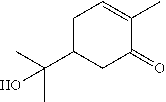 |
| Formula (XI) | 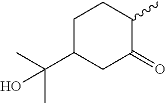 |
| Formula (XII) | 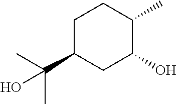 |
| Formula (XIII) | 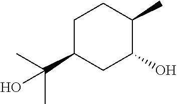 |
| Formula (XIV) | 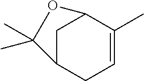 |
| Formula (XV) | 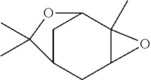 |

The sustained-release pellet comprises a second immediate-release pellet and a first sustained-release layer coated on the second immediate-release pellet, which is composed of a first sustained-release composition. The second immediate-release pellet is composed of a second medicated mixture, and the second medicated mixture comprises: a second compound, or its geometric isomer, enantiomer, diastereomer or pharmaceutically acceptable salt; and a second excipient composition, wherein a content of the second compound in the second medicated mixture is 5-85 wt %, and the second compound comprises sobrerol having the Formula (I) shown above or a sobrerol derivative having a formula selected from the group consisting of Formula (II)-Formula (XV) shown above. The first sustained-release composition comprises a first water-insoluble polymer and a first water-soluble binder. Moreover, the enteric-release pellet comprises a third immediate-release pellet and a first enteric-release layer coated on the third immediate-release pellet, which is composed of a first enteric-release composition. The third immediate-release pellet is composed of a third medicated mixture, and the third medicated mixture comprises: a third compound, or its geometric isomer, enantiomer, diastereomer or pharmaceutically acceptable salt; and a third excipient composition, wherein a content of the third compound in the third medicated mixture is 5-85 wt %, and the third compound comprises sobrerol having the Formula (I) shown above or a sobrerol derivative having a formula selected from the group consisting of Formula (II)-Formula (XV) shown above. The first enteric-release composition comprises a first enteric-release substance with pH-dependent solubility and a first anti-adherent plasticizer. Furthermore, the enteric coated sustained-release pellet comprises: a fourth immediate-release pellet; a second sustained-release layer coated on the fourth immediate-release pellet, which is composed of a second sustained-release composition; and a second enteric-release layer coated on the second sustained-release layer, which is composed of a second enteric-release composition. The fourth immediate-release pellet is composed of a fourth medicated mixture, and the fourth medicated mixture comprises: a fourth compound, or its geometric isomer, enantiomer, diastereomer or pharmaceutically acceptable salt; and a fourth excipient composition, wherein a content of the fourth compound in the fourth medicated mixture is 5-85 wt %, and the fourth compound comprises sobrerol having the Formula (I) shown above or a sobrerol derivative having a formula selected from the group consisting of Formula (II)-Formula (XV) shown above. In addition, the second sustained-release composition comprises a second water-insoluble polymer, and a second water-soluble binder. In addition, the second enteric-release composition comprises a second enteric-release substance with pH-dependent solubility and a second anti-adherent plasticizer.

The present disclosure also provides an immediate-release pellet, consisting of a medicated mixture, wherein the medicated mixture comprises: a compound, or its geometric isomer, enantiomer, diastereomer or pharmaceutically acceptable salt; and an excipient composition, which comprises a filler. A content of the compound in the medicated mixture is 5-85 wt %, and the compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 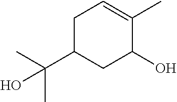 |
| Formula (II) | 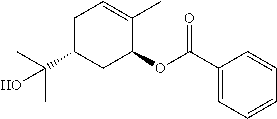 [SMILES: CC1=CC[C@@H](C(C)(C)O)C[C@@H]1OC(=O)c1ccccc1] |
| Formula (III) | 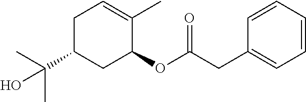 [SMILES: CC1=CC[C@@H](C(C)(C)O)C[C@@H]1OC(=O)Cc1ccccc1] |
| Formula (IV) | 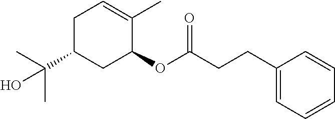 [SMILES: CC1=CC[C@@H](C(C)(C)O)C[C@@H]1OC(=O)CCc1ccccc1] |
| Formula (V) | 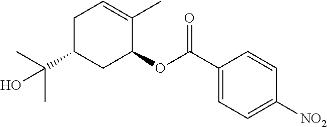 [SMILES: CC1=CC[C@@H](C(C)(C)O)C[C@@H]1OC(=O)c1ccc([N+](=O)[O-])cc1] |
| Formula (VI) | 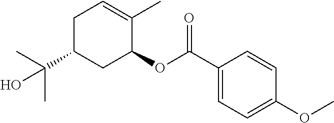 [SMILES: COc1ccc(C(=O)O[C@H]2C[C@H](C(C)(C)O)CC=C2C)cc1] |
| Formula (VII) | 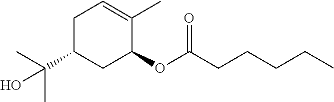 [SMILES: CCCCCC(=O)O[C@H]1C[C@H](C(C)(C)O)CC=C1C] |
| Formula (VIII) | 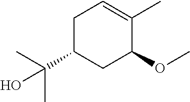 |
| Formula (IX) | 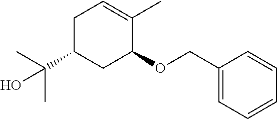 [SMILES: CC1=CC[C@@H](C(C)(C)O)C[C@@H]1OCc1ccccc1] |
| Formula (X) | 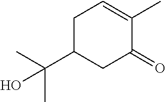 |
| Formula (XI) | 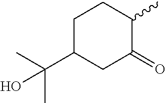 |
| Formula (XII) | 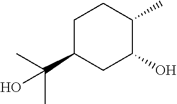 |
| Formula (XIII) | 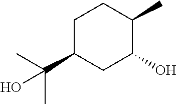 |
| Formula (XIV) | 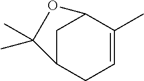 |
| Formula (XV) | 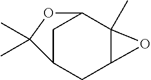 |

Moreover, a weight ratio of the compound to the excipient composition is 1:0.3-5.0, a weight ratio of the compound to the filler is 1:0.15-5.00, and a particle size of the immediate-release pellet is 360-1100 μm.

The present disclosure further provides sustained-release pellet, comprising: an immediate-release pellet; and a sustained-release composition coated on the immediate-release pellet to constitute a sustained-release layer. The immediate-release pellet is composed of a medicated mixture, and the medicated mixture comprises: a compound, or its geometric isomer, enantiomer, diastereomer or pharmaceutically acceptable salt; and an excipient composition. The compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 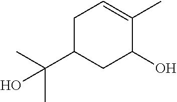 |
| Formula (II) | 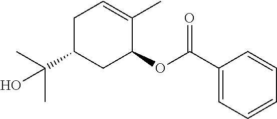 [SMILES: CC1=CC[C@@H](C(C)(C)O)C[C@@H]1OC(=O)c1ccccc1] |
| Formula (III) | 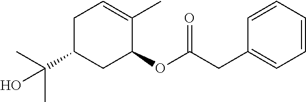 [SMILES: CC1=CC[C@@H](C(C)(C)O)C[C@@H]1OC(=O)Cc1ccccc1] |
| Formula (IV) | 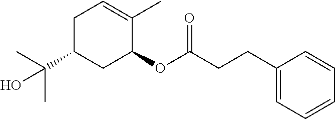 [SMILES: CC1=CC[C@@H](C(C)(C)O)C[C@@H]1OC(=O)CCc1ccccc1] |
| Formula (V) | 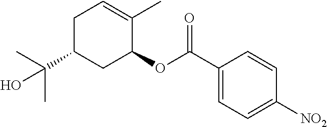 [SMILES: CC1=CC[C@@H](C(C)(C)O)C[C@@H]1OC(=O)c1ccc([N+](=O)[O-])cc1] |
| Formula (VI) | 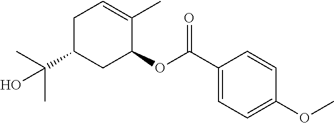 [SMILES: COc1ccc(C(=O)O[C@H]2C[C@H](C(C)(C)O)CC=C2C)cc1] |
| Formula (VII) | 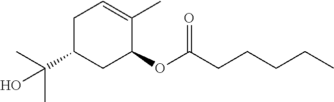 [SMILES: CCCCCC(=O)O[C@H]1C[C@H](C(C)(C)O)CC=C1C] |
| Formula (VIII) | 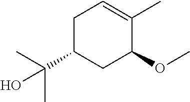 |
| Formula (IX) | 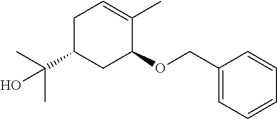 [SMILES: CC1=CC[C@@H](C(C)(C)O)C[C@@H]1OCc1ccccc1] |
| Formula (X) | 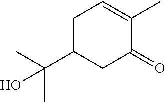 |
| Formula (XI) | 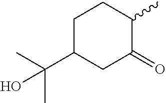 |
| Formula (XII) | 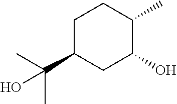 |
| Formula (XIII) | 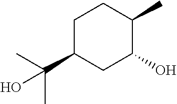 |
| Formula (XIV) | 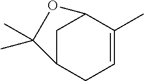 |
| Formula (XV) | 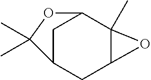 |

Moreover, the sustained-release composition comprises a water-insoluble polymer and a water-soluble binder. In addition, a content of the compound in the medicated mixture is 5-85 wt %, a weight ratio of the compound to the excipient composition is 1:0.4-5.5, and a weight ratio of the compound to the filler is 1:0.15-5.00, a weight ratio of the immediate-release pellet to the sustained-release composition is 1:0.08-0.60, and a weight ratio of the second immediate-release pellet, the water-insoluble polymer and the water-soluble binder is 1:0.040-0.100:0.009-0.030, and a particle size of the sustained-release pellet is 425-1200 μm.

The present disclosure further provides an enteric-release pellet comprising: an immediate-release pellet; and an enteric-release composition coated on the immediate-release pellet to constitute an enteric-release layer. The immediate-release pellet is composed of a medicated mixture, and the medicated mixture comprises: a compound, or its geometric isomer, enantiomer, diastereomer or pharmaceutically acceptable salt; and an excipient composition. The compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 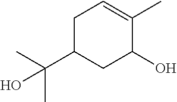 |
| Formula (II) | 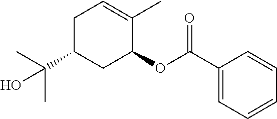 [SMILES: CC1=CC[C@@H](C(C)(C)O)C[C@@H]1OC(=O)c1ccccc1] |
| Formula (III) | 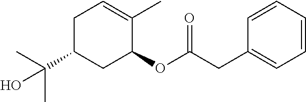 [SMILES: CC1=CC[C@@H](C(C)(C)O)C[C@@H]1OC(=O)Cc1ccccc1] |
| Formula (IV) | 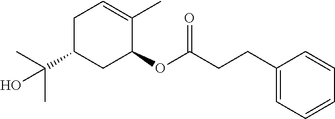 [SMILES: CC1=CC[C@@H](C(C)(C)O)C[C@@H]1OC(=O)CCc1ccccc1] |
| Formula (V) | 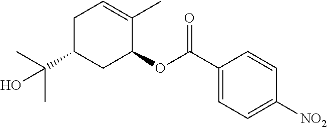 [SMILES: CC1=CC[C@@H](C(C)(C)O)C[C@@H]1OC(=O)c1ccc([N+](=O)[O-])cc1] |
| Formula (VI) | 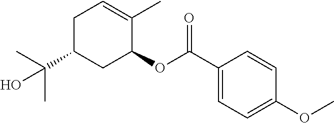 [SMILES: COc1ccc(C(=O)O[C@H]2C[C@H](C(C)(C)O)CC=C2C)cc1] |
| Formula (VII) | 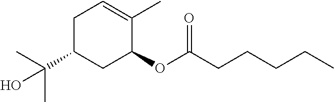 [SMILES: CCCCCC(=O)O[C@H]1C[C@H](C(C)(C)O)CC=C1C] |
| Formula (VIII) | 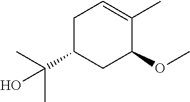 |
| Formula (IX) | 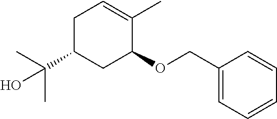 [SMILES: CC1=CC[C@@H](C(C)(C)O)C[C@@H]1OCc1ccccc1] |
| Formula (X) | 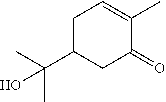 |
| Formula (XI) | 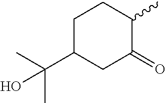 |
| Formula (XII) | 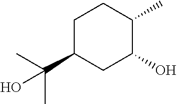 |
| Formula (XIII) | 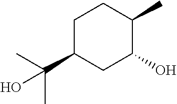 |
| Formula (XIV) | 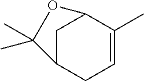 |
| Formula (XV) | 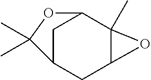 |

Furthermore, the enteric-release composition comprises an enteric-release substance with pH-dependent solubility and an anti-adherent plasticizer. In addition, a content of the compound in the medicated mixture is 5-85 wt %, a weight ratio of the compound to the excipient composition is 1:0.65-6.00, and a weight ratio of the compound to the filler is 1:0.15-5.00, a weight ratio of the immediate-release pellet to the enteric-release composition is 1:0.20-0.40, and a weight ratio of the immediate-release pellet, the enteric-release substance with pH-dependent solubility and the anti-adherent plasticizer is 1:0.01-1:0.005-0.5, and a particle size of the enteric-release pellet is 465-1380 μm.

The present disclosure further provides a method for treating an autoimmune neurological disease and/or a neurodegenerative disease. The method comprises administering an effective amount of the multi-particulate pharmaceutical composition mentioned above.

Furthermore, the present disclosure also provides another method for treating an autoimmune neurological disease and/or a neurodegenerative disease. The method comprises administering an effective amount of the immediate-release pellet mentioned above, the sustained-release pellet mentioned above or the enteric-release pellet mentioned above.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows the results of the solubility test of sobrerol in dissolution medium with different pH and purified water;

FIG. 2A shows the dissolution curves of 20% sobrerol immediate-release pellets in dissolution medium with different pH and purified water;

FIG. 2B shows the dissolution curves of 20% sobrerol immediate-release pellets and 70% sobrerol immediate-release pellets in purified water water;

FIG. 3 shows the linear relationship between the sobrerol in vivo absorption percentage curve and the dissolution curve of 20% sobrerol immediate-release pellets in purified water water;

FIG. 4A shows the dissolution curves of the sustained-release pellets with different the sustained-release layers formed by different formulations in purified water water;

FIG. 4B shows the dissolution curves of the sustained-release pellets SR3 in dissolution medium with different pH;

FIG. 5A shows the dissolution curves of the enteric-release pellets in dissolution testing with different pH stages;

FIG. 5B shows the dissolution curves of the enteric-release pellets and the enteric coated sustained-release pellets in dissolution testing with different pH stages;

FIG. 6A shows a comparison of the dissolution curves of the sobrerol immediate-release pellets, the sustained-release pellets SR3 and the combination of sobrerol immediate-release pellets and the sustained-release pellets SR3;

FIG. 6B shows a comparison of the dissolution curves of the immediate-release pellets, the enteric-release pellets, and a combination of the sobrerol immediate-release pellets and the enteric-release pellets;

FIG. 6C shows a comparison of the dissolution curves of the sobrerol sustained-release pellets, the enteric-release pellets, and the combination of the sobrerol sustained-release pellets and the enteric-release pellets; and

FIG. 6D shows a comparison of the dissolution curves of the sobrerol immediate-release pellets, the sustained-release pellets, the enteric-release pellets, and a combination of the sobrerol immediate-release pellets, the sustained-release pellets and the enteric-release pellets.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure provides a multi-particulate pharmaceutical composition. The multi-particulate pharmaceutical composition of the present disclosure may have an effect of being capable of in vivo rapidly increasing the drug concentration in the blood and stably releasing the drug for long-term, an effect of being capable of in vivo making the drug contained therein reach at least two peak concentrations over time to reduce the number of times the drug is taken, an effect of being capable of in vivo slowing down the decrease level of a sustained release drug concentration in the blood during the period of rapid drug metabolism to avoid premature decrease in drug concentration, and an effect being capable of in vivo increasing the trough concentration between at least two peak concentrations reached by the drug contained therein over time, etc.

Furthermore, for a subject in need to be administered the drug contained in the multi-particulate pharmaceutical composition of the present disclosure, the multi-particulate pharmaceutical composition of the present disclosure can effectively maintain the concentration of the drug, such as the concentration of the drug in the blood, in the subject for a long time within a desired concentration range, and a single administration of the multi-particulate pharmaceutical composition of the present disclosure can have an effect of multiple administrations of the drug, and thus the multi-particulate pharmaceutical composition of the present disclosure can achieve an effect of reducing the frequency and/or dosage of the administration. The subject mentioned herein may comprise, but is not limited to, a vertebrate. The vertebrate mentioned above may comprise a fish, an amphibian, a reptile, a bird, or a mammal, but it is not limited thereto. Examples of mammals may comprise, but are not limited to, a human, an orangutan, a monkey, a horse, a donkey, a dog, a cat, a rabbit, a guinea pig, a rat, and a mouse. In one embodiment, the subject mentioned herein is a human.

With regard to the components for the multi-particulate pharmaceutical composition of the present disclosure mentioned above, the detailed description is as follows.

The multi-particulate pharmaceutical composition of the present disclosure mentioned above, may comprise, but is not limited to, mixture pellets.

The mixture pellets mentioned above may comprise at least two of the following pellets: a first immediate-release pellet, a first sustained-release pellet, an enteric-release pellet, an enteric coated sustained-release pellet, etc., but it is not limited thereto.

For the mixture pellets of the multi-particulate pharmaceutical composition of the present disclosure, the first immediate-release pellet mentioned above, may be composed of a first medicated mixture. A particle size of the first immediate-release pellet may be about 360-1100 μm, such as about 500-700 μm, about 700-900 μm, but it is not limited thereto. In one embodiment, the particle size of the first immediate-release pellet may be about 600-1000 μm.

Moreover, the first medicated mixture may comprise, a first compound, or its geometric isomer, enantiomer, diastereomer or pharmaceutically acceptable salt and a first excipient composition, but it is not limited thereto.

Furthermore, the foregoing first compound may comprise, but is not limited to, sobrerol having the Formula (I) shown in the following or a sobrerol derivative having a formula selected from the group consisting of Formula (II)-Formula (XV) shown in the following:

| Formula | Compound |
|---|---|
| Formula (I) | 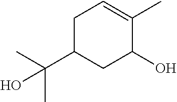 |
| Formula (II) | 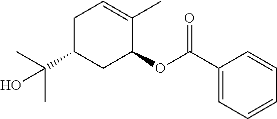 [SMILES: CC1=CC[C@@H](C(C)(C)O)C[C@@H]1OC(=O)c1ccccc1] |
| Formula (III) | 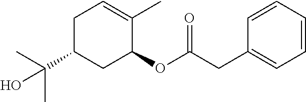 [SMILES: CC1=CC[C@@H](C(C)(C)O)C[C@@H]1OC(=O)Cc1ccccc1] |
| Formula (IV) | 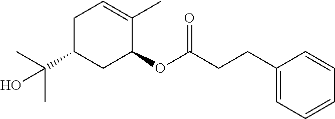 [SMILES: CC1=CC[C@@H](C(C)(C)O)C[C@@H]1OC(=O)CCc1ccccc1] |
| Formula (V) | 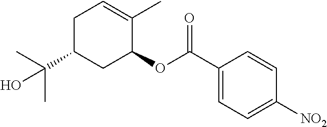 [SMILES: CC1=CC[C@@H](C(C)(C)O)C[C@@H]1OC(=O)c1ccc([N+](=O)[O-])cc1] |
| Formula (VI) | 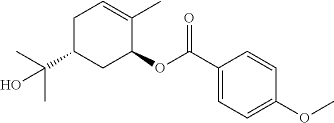 [SMILES: COc1ccc(C(=O)O[C@H]2C[C@H](C(C)(C)O)CC=C2C)cc1] |
| Formula (VII) | 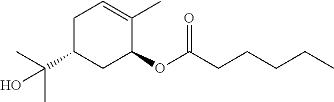 [SMILES: CCCCCC(=O)O[C@H]1C[C@H](C(C)(C)O)CC=C1C] |
| Formula (VIII) | 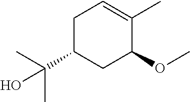 |
| Formula (IX) | 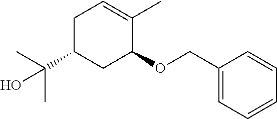 [SMILES: CC1=CC[C@@H](C(C)(C)O)C[C@@H]1OCc1ccccc1] |
| Formula (X) | 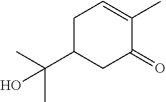 |
| Formula (XI) | 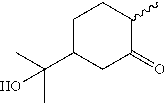 |
| Formula (XII) | 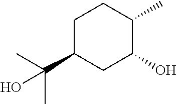 |
| Formula (XIII) | 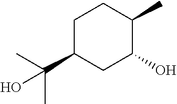 |
| Formula (XIV) | 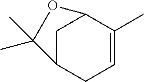 |
| Formula (XV) | 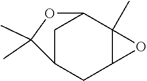 |

In one embodiment, the foregoing first compound may be sobrerol. The sobrerol or sobrerol derivative may have an effect of treating and/or alleviating and/or preventing an autoimmune neurological disease and/or a neurodegenerative disease, but it is not limited thereto.

In one embodiment, a content of the first compound in the first medicated mixture may be about 5-85 wt %, such as about 10-85 wt %, about 15-85 wt %, about 20-85 wt %, about 5-80 wt %, about 5-75 wt %, about 5-70 wt %, about 10-80 wt %, about 15-75 wt %, about 20-70 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, but it is not limited thereto. In one specific embodiment, the content of the first compound in the first medicated mixture may be about 20 wt %. In another specific embodiment, the content of the first compound in the first medicated mixture may be about 70 wt %.

Moreover, in the first medicated mixture mentioned above, a weight ratio of the first compound to the first excipient composition may be about 1:0.3-5.0, such as about 1:0.5-4.5, 1:0.7-4.2, about 1:0.8-4.0, about 1:1.0-3.5, about 1:0.3-0.7, about 1:0.7-1.0, about 1:1.0-1.5, about 1:1.5-2.5, about 1:2.5-3.5, about 1:3.5-5.0, but it is not limited thereto.

In one embodiment, the first excipient composition mentioned above may comprise a filler, but it is not limited thereto. Examples of the filler, may comprise, but are not limited to, at least one of the following ingredients: microcrystalline cellulose, lactose, starch, corn starch, mannitol, calcium hydrogen phosphate, dicalcium phosphate, etc. In one specific embodiment, the filler mentioned above may comprise microcrystalline cellulose. In another specific embodiment, the filler mentioned above is microcrystalline cellulose.

Furthermore, in one specific embodiment, in the first immediate-release pellet, the first compound mentioned above is sobrerol, the first excipient composition mentioned above consists of the filler, and the filler is microcrystalline cellulose.

In another embodiment, the first excipient composition mentioned above, in addition to the filler, may further comprise a binder and/or an anti-adherent.

Examples of the foregoing binder, may comprise, but are not limited to, at least one of the following ingredients: hydroxypropyl methyl cellulose, hydroxypropyl cellulose, polyvinylpyrrolidone, polyvinyl alcohol, etc. Meanwhile, the foregoing anti-adherent may comprise at least one of the following ingredients: talc powder, stearic acid, stearate, silicon dioxide in powder form or colloidal form, etc., but it is not limited thereto.

In one specific embodiment, the first excipient composition comprises the filler and the binder. Meanwhile, in this specific embodiment, a weight ratio of the filler to the binder in the first excipient composition may be about 1:0.001-0.70, such as about 1:0.005-0.05, about 1:0.01-0.25, about 1:0.05-0.5, about 1:0.20, but it is not limited thereto.

In another specific embodiment, the first excipient composition comprises the filler and the anti-adherent. Moreover, in this specific embodiment, a weight ratio of the filler to the anti-adherent in the first excipient composition may be about 1:0.001-0.35, such as about 1:0.005-0.05, about 1:0.01-0.2, about 1:0.05-0.1, about 1:0.04, but it is not limited thereto.

In another specific embodiment, the first excipient composition comprises the filler, the anti-adherent and the anti-adherent. Moreover, in this specific embodiment, a weight ratio of the filler to the binder to the anti-adherent in the first excipient composition may be about 1:0.001-0.70:0.001-0.35, such as about 1:0.001-0.05:0.001-0.003, about 1:0.001:0.001, about 1:0.25:0.01, but it is not limited thereto.

In one specific embodiment, for the first immediate-release pellet mentioned above, the weight ratio of the first compound to the first excipient composition may be about 1:0.42-4.00, the weight ratio of the first compound to the first filler in the first excipient composition is about 1:0.42-4.00, and the particle size of the first immediate-release pellet may be about 600-1000 μm. Furthermore, in this specific embodiment, the first compound is sobrerol, the filler in the first excipient composition is microcrystalline cellulose, and the content of sobrerol in the first medicated mixture composing the first immediate-release pellet is about 20-70 wt %.

For the mixture pellets of the multi-particulate pharmaceutical composition of the present disclosure, the sustained-release pellet mentioned above may comprise, but is not limited to, a second immediate-release pellet and a first sustained-release layer coated on the second immediate-release pellet, which may be composed of a first sustained-release composition. A weight ratio of the second immediate-release pellet to the first sustained-release composition may be about 1:0.01-0.2, such as about 1:0.01-0.15, about 1:0.01-0.1, about 1:0.01-0.05, 1:0.05-0.2, 1:0.1-0.2, 1:0.15-0.2, about 1:0.69, about 1:0.083, about 1:0.097, but it is not limited thereto. Moreover, a particle size of the sustained-release pellet may be about 425-1200 μm, such as about 450-650 μm, about 700-1000 μm, but it is not limited thereto. In one embodiment, the particle size of the sustained-release pellet may be about 600-1050 μm.

In addition, the foregoing second immediate-release pellet in the foregoing sustained-release pellet, may be composed of a second medicated mixture. A particle size of the second immediate-release pellet may be about 360-1100 μm, such as about 500-700 μm, about 700-900 μm, but it is not limited thereto. In one embodiment, the particle size of the second immediate-release pellet may be about 600-1000 μm.

The second medicated mixture may comprise, a second compound, or its geometric isomer, enantiomer, diastereomer or pharmaceutically acceptable salt and a second excipient composition, but it is not limited thereto.

Meanwhile, the second compound mentioned above may comprise, sobrerol having the Formula (I) shown above or a sobrerol derivative having one of Formula (II)-Formula (XV) shown in the following, but it is not limited thereto. In one embodiment, the second compound mentioned above may be sobrerol.

In one embodiment, a content of the second compound in the second medicated mixture may be about 5-85 wt %, such as about 10-85 wt %, about 15-85 wt %, about 20-85 wt %, about 5-80 wt %, about 5-75 wt %, about 5-70 wt %, about 10-80 wt %, about 15-75 wt %, about 20-70 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, but it is not limited thereto. In one specific embodiment, the content of the second compound in the second medicated mixture may be about 20 wt %. In another specific embodiment, the content of the second compound in the second medicated mixture may be about 70 wt %.

Moreover, in the second medicated mixture mentioned above, a weight ratio of the second compound to the second excipient composition may be about 1:0.3-5.0, such as about 1:0.5-4.5, about 1:0.7-4.2, about 1:0.8-4.0, about 1:1.0-3.5, about 1:0.3-0.7, about 1:0.7-1.0, about 1:1.0-1.5, about 1:1.5-2.5, about 1:2.5-3.5, about 1:3.5-5.0, but it is not limited thereto.

With regard to the second excipient composition, all related description thereof can be the same as all related description for the foregoing first excipient composition, and thus is not repeated therein.

Furthermore, in one specific embodiment, for the second immediate-release pellet mentioned above, the weight ratio of the second compound to the second excipient composition may be about 1:0.42-4.00, the weight ratio of the second compound to the second filler in the second excipient composition is about 1:0.42-4.00, and the particle size of the second immediate-release pellet may be about 600-1000 μm. In addition, in this specific embodiment, the second compound is sobrerol, and the filler in the second excipient composition is microcrystalline cellulose, and the content of sobrerol in the second medicated mixture composing the second immediate-release pellet is about 20-70 wt %.

The first sustained-release composition composing the first sustained-release layer of the sustained-release pellet may comprise, a first water-insoluble polymer and a first water-soluble binder, but it is not limited thereto. A weight ratio of first water-insoluble polymer to the first water-soluble binder may be about 1:0.05-1, such as about 1:0.1-1, about 1:0.5-1, about 1:0.05-0.5, about 1:0.05-0.1, about 1:0.20, about 1:0.25, about 1:0.30, but it is not limited thereto. Moreover, a weight ratio of the second immediate-release pellet to the first water-insoluble polymer to the first water-soluble binder may be about 1:0.01-0.1:0.005-0.05, such as about 1:0.05-0.1:0.01-0.05, about 1:0.01-0.05:0.005-0.01, about 1:0.053-0.077:0.013-0.019, but it is not limited thereto.

Examples of the first water-insoluble polymer mentioned above, may comprise, but are not limited to, at least one of the following ingredients: ethyl cellulose, ethyl acrylate-methyl methacrylate-trimethyl ammonium chloride ethyl methacrylate copolymer, methyl methacrylate-ethyl acrylate copolymer, hydrogenated castor oil, hydrogenated coconut oil, stearic acid, stearyl alcohol, etc. In one embodiment, first water-insoluble polymer in the first sustained-release composition composing the first sustained-release layer may be ethyl cellulose.

Moreover, examples of the foregoing first water-soluble binder, may comprise, but are not limited to, at least one of the following ingredients: hydroxypropyl methyl cellulose, hydroxypropyl cellulose, polyvinylpyrrolidone, polyvinyl alcohol, etc. In one embodiment, the water-soluble binder in the first sustained-release composition composing first sustained-release layer may be hydroxypropyl methyl cellulose.

In one specific embodiment, for the sustained-release pellet mentioned above, the content of the second compound in the second medicated mixture may be 5-85 wt %, the weight ratio of the second compound to the second excipient composition may be about 1:0.42-4.00, the weight ratio of the second compound to the second filler in the second excipient composition is 1:0.42-4.00, the weight ratio of the second immediate-release pellet to the sustained-release composition is 1:0.05-0.15, the weight ratio of the second immediate-release pellet to the first water-insoluble polymer in the first sustained-release composition to the first water-soluble binder in the first sustained-release composition is about 1:0.040-0.1000:0.009-0.030, and the particle size of the sustained-release pellet may be about 425-1200 μm. Furthermore, in this specific embodiment, the second compound is sobrerol, and the filler in the second excipient composition is microcrystalline cellulose, and the content of sobrerol in the second medicated mixture comprising the second immediate-release pellet is about 20-70 wt %, and the first water-insoluble polymer in the first sustained-release composition is ethyl cellulose, and the first water-soluble binder of the first sustained-release composition is hydroxypropyl methyl cellulose.

Moreover, in one embodiment, the sustained-release pellet mentioned above, in addition to the second immediate-release pellet and the first sustained-release layer mentioned above, may further comprise a coating layer coated on first sustained-release layer, which may be composed of a coating composition. The coating layer may occupy about 0.01-20 wt %, such as about 0.01-5 wt %, about 1-10 wt %, about 12.5 wt %, about 10 wt % by weight of the sustained-release pellet, but it is not limited thereto.

The purpose of the coating layer is to prevent a controlled-release layer, such as a sustained-release layer, a enteric-release layer, from being damaged or corroded, and losing the ability to control release, and thus for the ingredients of the coating composition composing the coating layer, an excipient for a common coating can be chosen, such as hydroxypropyl methyl cellulose, polyethylene glycol, lactose, talc powder, titanium dioxide or any combination thereof, and a commercial composition also can be considered, such as Opadry film coating or Kollicoat film coating, but it is not limited thereto.

In addition, for the mixture pellets of the multi-particulate pharmaceutical composition of the present disclosure, the enteric-release pellet mentioned above may comprise, but is not limited to, a third immediate-release pellet and a first enteric-release layer coated on the third immediate-release pellet, which may be composed of a first enteric-release composition. A weight ratio of the third immediate-release pellet to the first enteric-release composition may be about 1:0.1-1.5, such as about 1:0.5-1.5, about 1:1-1.5, about 1:0.1-1, about 1:0.1-0.5, about 1:0.88, but it is not limited thereto. Moreover, a particle size of the enteric-release pellet may be about 465-1380 μm, such as about 750-950 μm, about 900-1200 μm, but it is not limited thereto. In one embodiment, a particle of the enteric-release pellet may be about 800-1300 μm.

Meanwhile, the foregoing third immediate-release pellet in the foregoing enteric-release pellet, may be composed of a third medicated mixture. A particle size of the third immediate-release pellet may be about 360-1100 μm, such as about 500-700 μm, about 700-900 μm, but it is not limited thereto. In one embodiment, the particle size of the third immediate-release pellet may be about 600-1000 μm.

The third medicated mixture may comprise, a third compound, or its geometric isomer, enantiomer, diastereomer or pharmaceutically acceptable salt and a third excipient composition, but it is not limited thereto.

Moreover, the third compound mentioned above may comprise, sobrerol having the Formula (I) shown above or a sobrerol derivative having one of Formula (II)-Formula (XV) shown in the following, but it is not limited thereto. In one embodiment, the third compound mentioned above may be sobrerol.

In one embodiment, a content of the third compound in the third medicated mixture may be about 5-85 wt %, such as about 10-85 wt %, about 15-85 wt %, about 20-85 wt %, about 5-80 wt %, about 5-75 wt %, about 5-70 wt %, about 10-80 wt %, about 15-75 wt %, about 20-70 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, but it is not limited thereto. In one specific embodiment, the content of the third compound in the third medicated mixture may be about 20 wt %. In another specific embodiment, the content of the third compound in the third medicated mixture may be about 70 wt %.

Moreover, in the third medicated mixture mentioned above, a weight ratio of the third compound to the third excipient composition may be about 1:0.3-5.0, such as about 1:0.5-4.5, about 1:0.7-4.2, about 1:0.8-4.0, about 1:1.0-3.5, about 1:0.3-0.7, about 1:0.7-1.0, about 1:1.0-1.5, about 1:1.5-2.5, about 1:2.5-3.5, about 1:3.5-5.0, but it is not limited thereto.

With regard to the third excipient composition, all related description thereof can be the same as all related description for the first excipient composition mentioned above, and thus is not repeated therein.

Furthermore, in one specific embodiment, for the third immediate-release pellet mentioned above, the weight ratio of the third compound to the third excipient composition may be about 1:0.42-4.00, the weight ratio of the third compound to the third filler in the third excipient composition is about 1:0.42-4.00, and the particle size of the third immediate-release pellet may be about 600-1000 μm. Moreover, in this specific embodiment, the third compound is sobrerol, and the filler in the third excipient composition is microcrystalline cellulose, and the content of sobrerol in the third medicated mixture composing the third immediate-release pellet is about 20-70 wt %.

Moreover, the first enteric-release composition composing the first enteric-release layer of the enteric-release pellet may comprise, a first enteric-release substance with pH-dependent solubility and the first anti-adherent plasticizer, but it is not limited thereto. A weight ratio of the first enteric-release substance with pH-dependent solubility to the first anti-adherent plasticizer may be about 1:0.01-1, such as about 1:0.05-1, about 1:0.1-1, about 1:0.05-0.5, about 1:0.2, about 1:0.25, about 1:0.3, but it is not limited thereto. Furthermore, a weight ratio of the third immediate-release pellet to the first enteric-release substance with pH-dependent solubility to the first anti-adherent plasticizer may be about 1:0.01-1:0.005-0.5, such as about 1:0.05-1:0.01-0.5, about 1:0.1-1:0.05-1, about 1:0.7:0.18, but it is not limited thereto.

Examples of the foregoing first enteric-release substance with pH-dependent solubility, may comprise, but is not limited to, at least one of the following ingredients: an enteric acrylic copolymer, an enteric cellulose derivative, an enteric starch derivative, saturated fatty acid, etc. The enteric acrylic copolymer may comprise, methacrylic acid-methyl methacrylate copolymer, methacrylic acid-ethyl acrylate copolymer, etc., or any combination thereof, but it is not limited thereto. The enteric cellulose derivative may comprise, but is not limited to, hydroxypropyl methylcellulose phthalate, hydroxypropyl methylcellulose acetate succinate, carboxymethyl ethyl cellulose, cellulose acetate trimellitate, cellulose acetate phthalate, cellulose acetate succinate, or any combination thereof. The enteric starch derivative may comprise, starch acetate phthalate, amylose acetate phthalate, etc., or any combination thereof, but it is not limited thereto. Saturated fatty acid may comprise, but is not limited to, stearic acid, Palmitic acid, myristic acid, lauric acid, etc., or any combination thereof.

The first anti-adherent plasticizer mentioned above, may comprise, but is not limited to, at least one of the following ingredients: glyceryl monostearate, polysorbate 80 and triethyl citrate.

In one specific embodiment, for the enteric-release pellet mentioned above, the content of the third compound in the third medicated mixture may be 5-85 wt %, the weight ratio of the third compound to the third excipient composition may be about 1:0.42-4.00, the weight ratio of the third compound to the filler in the third excipient composition is about 1:0.42-4.00, the weight ratio of the third immediate-release pellet to the first enteric-release composition is about 1:0.15-0.50, the weight ratio of the third immediate-release pellet to the first enteric-release substance with pH-dependent solubility in the first enteric-release composition to the first anti-adherent plasticizer in the first enteric-release composition is about 1:0.05-1:0.01-0.5, and the particle size of the enteric-release pellet may be about 465-1380 μm. Furthermore, in this specific embodiment, the third compound is sobrerol, and the filler in the third excipient composition is microcrystalline cellulose, and a content sobrerol in the third medicated mixture composing the third immediate-release pellet is about 20-70 wt %, and the first enteric-release substance with pH-dependent solubility in the first enteric-release composition comprises ethacrylic acid-ethyl acrylate copolymer, and the first anti-adherent plasticizer in the first enteric-release composition comprises glyceryl monostearate, polysorbate 80 and triethyl citrate.

Moreover, in one embodiment, the enteric-release pellet mentioned above, in addition to the third immediate-release pellet and the first enteric-release layer mentioned above, may further comprise a coating layer coated on the first enteric-release layer, which may be composed of a coating composition. The coating layer may occupy about 0.01-20 wt %, such as about 0.01-5 wt %, about 1-10 wt %, about 12.5 wt %, about 10 wt % by weight of the sustained-release pellet, but it is not limited thereto.

The relevant description of the coating layer of the enteric-release pellet may be the same as the coating layer of the sustained-release pellet, and thus not repeated herein.

Moreover, for the mixture pellets of the multi-particulate pharmaceutical composition of the present disclosure, the enteric coated sustained-release pellet mentioned above may comprise, but is not limited to, a fourth immediate-release pellet, a second sustained-release layer coated on the fourth immediate-release pellet, which may be composed of a second sustained-release composition, and a second enteric-release layer coated on the second sustained-release layer, which is composed of a second enteric-release composition. A weight ratio of the fourth immediate-release pellet coated with the second sustained-release layer to the second enteric-release composition may be about 1:0.1-1.5, such as about 1:0.5-1.5, about 1:1-1.5, about 1:0.1-1, about 1:0.1-0.5, about 1:0.88, but it is not limited thereto. Moreover, a particle size of the enteric coated sustained-release pellet may be about 465-1450 μm, such as about 600-900 μm, about 750-1150 μm, but it is not limited thereto. In one embodiment, the particle size of the enteric coated sustained-release pellet may be about 800-1300 μm.

The foregoing fourth immediate-release pellet in the foregoing enteric coated sustained-release pellet, may be composed of a fourth medicated mixture. A particle size of the fourth immediate-release pellet may be about 360-1100 μm, such as about 500-700 μm, about 700-900 μm, but it is not limited thereto. In one embodiment, the particle size of the fourth immediate-release pellet may be about 600-1000 μm.

The fourth medicated mixture may comprise, a fourth compound, or its geometric isomer, enantiomer, diastereomer or pharmaceutically acceptable salt and a fourth excipient composition, but it is not limited thereto.

The fourth compound mentioned above may comprise, sobrerol having the Formula (I) shown above or a sobrerol derivative having one of Formula (II)-Formula (XV) shown in the following, but it is not limited thereto. In one embodiment, the second compound mentioned above may be sobrerol.

In one embodiment, a content of the fourth compound in the fourth medicated mixture may be about 5-85 wt %, such as about 10-85 wt %, about 15-85 wt %, about 20-85 wt %, about 5-80 wt %, about 5-75 wt %, about 5-70 wt %, about 10-80 wt %, about 15-75 wt %, about 20-70 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, but it is not limited thereto. In one specific embodiment, the content of the fourth compound in the fourth medicated mixture may be about 20 wt %. In another specific embodiment, the content of the fourth compound in the fourth medicated mixture may be about 70 wt %.

Furthermore, in the fourth medicated mixture mentioned above, a weight ratio of the fourth compound to the fourth excipient composition may be about 1:0.3-5.0, such as about 1:0.5-4.5, about 1:0.7-4.2, about 1:0.8-4.0, about 1:1.0-3.5, about 1:0.3-0.7, about 1:0.7-1.0, about 1:1.0-1.5, about 1:1.5-2.5, about 1:2.5-3.5, about 1:3.5-5.0, but it is not limited thereto.

With regard to the fourth excipient composition, all related description thereof can be the same as all related description for the first excipient composition mentioned above, and thus is not repeated therein.

In addition, in one specific embodiment, for the fourth immediate-release pellet mentioned above, the weight ratio of the fourth compound to the fourth excipient composition may be about 1:0.42-4.00, the weight ratio of the fourth compound to the filler in the fourth excipient composition is about 1:0.42-4.00, and the particle size of the fourth immediate-release pellet may be about 600-1000 μm. Moreover, in this specific embodiment, the fourth compound is sobrerol, and the filler in the fourth excipient composition is microcrystalline cellulose, and the content of sobrerol in the fourth medicated mixture composing the fourth immediate-release pellet is about 20-70 wt %.

The second sustained-release composition composing the second sustained-release layer of the enteric coated sustained-release pellet may comprise, a second water-insoluble polymer and a second water-soluble binder, but it is not limited thereto. A weight ratio of the second water-insoluble polymer to the second water-soluble binder may be about 1:0.05-1, such as about 1:0.1-1, about 1:0.5-1, about 1:0.05-0.5, about 1:0.05-0.1, about 1:0.20, about 1:0.25, about 1:0.30, but it is not limited thereto. Moreover, a weight ratio of the fourth immediate-release pellet to the second water-insoluble polymer to the second water-soluble binder may be about 1:0.01-0.1:0.005-0.05, such as about 1:0.05-0.1:0.01-0.05, about 1:0.01-0.05:0.005-0.01, about 1:0.053-0.077:0.013-0.019, but it is not limited thereto.

With regard to the second water-insoluble polymer, all related description thereof can be the same as all related description for the first water-insoluble polymer mentioned above, and thus is not repeated therein. Similarly, with regard to the second water-insoluble polymer, all related description thereof can be the same as all related description for the first water-insoluble polymer mentioned above, and thus is not repeated therein.

The second enteric-release composition comprising the second enteric-release layer of the enteric coated sustained-release pellet may comprise, a second enteric-release substance with pH-dependent solubility and a second anti-adherent plasticizer, but it is not limited thereto. A weight ratio of the second enteric-release substance with pH-dependent solubility to the second anti-adherent plasticizer may be about 1:0.01-1, such as about 1:0.05-1, about 1:0.1-1, about 1:0.05-0.5, about 1:0.2, about 1:0.25, about 1:0.3, but it is not limited thereto. Moreover, a weight ratio of the fourth immediate-release pellet coated with the second sustained-release layer to the second enteric-release substance with pH-dependent solubility to the second anti-adherent plasticizer may be about 1:0.01-1:0.005-0.5, such as about 1:0.05-1:0.01-0.5, about 1:0.1-1:0.05-1, about 1:0.7:0.18, but it is not limited thereto.

With regard to the second enteric-release substance with pH-dependent solubility and the second anti-adherent plasticizer, all related description thereof can be the same as all related description for the first enteric-release substance with pH-dependent solubility and the first anti-adherent plasticizer mentioned above, and thus is not repeated therein.

In one specific embodiment, for the enteric sustained-release pellet mentioned above, the content of the fourth compound in the fourth medicated mixture may be 5-85 wt %, the weight ratio of the fourth compound to the fourth excipient composition may be about 1:0.42-4.00, the weight ratio of the fourth compound to the filler in the fourth excipient composition is about 1:0.42-4.00, the weight ratio of the fourth immediate-release pellet to the second sustained-release composition is about 1:0.05-0.15, the weight ratio of the fourth immediate-release pellet to the second water-insoluble polymer in the second sustained-release composition to the second water-soluble binder in the second sustained-release composition is about 1:0.040-0.100:0.009-0.030, the weight ratio of the fourth immediate-release pellet coated with the second sustained-release layer to the second enteric-release composition is about 1:0.15-0.50, the weight ratio of the fourth immediate-release pellet coated with the second sustained-release layer to the second enteric-release substance with pH-dependent solubility in the second enteric-release composition to the second anti-adherent plasticizer of the second enteric-release composition is about 1:0.05-1:0.01-0.5, and the particle size of the enteric coated sustained-release pellet may be about 800-1300 μm. Moreover, in this specific embodiment, the fourth compound is sobrerol, and the filler in the fourth excipient composition is microcrystalline cellulose, and the content of sobrerol in the fourth medicated mixture composing the fourth immediate-release pellet is about 20-70 wt %, and the second water-insoluble polymer in the second sustained-release composition is ethyl cellulose, and the first water-soluble binder in the second sustained-release composition is hydroxypropyl methyl cellulose, and the second enteric-release substance with pH-dependent solubility in the second enteric-release composition comprises methacrylic acid-ethyl acrylate copolymer, and the second anti-adherent plasticizer in the second enteric-release composition comprise glyceryl monostearate, polysorbate 80 and triethyl citrate.

Moreover, in one embodiment, the enteric sustained-release pellet mentioned above, in addition to the fourth immediate-release pellet, the second sustained-release layer and the second enteric-release layer mentioned above, may further comprise a coating layer coated on the second enteric-release layer, which may be composed of a coating composition. The coating layer may occupy about 0.01-20 wt %, such as about 0.01-5 wt %, about 1-10 wt %, about 12.5 wt %, about 10 wt % by weight of the sustained-release pellet, but it is not limited thereto.

The relevant description of the coating layer of the enteric coated sustained-release pellet may be the same as the coating layer of the sustained-release pellet, and this is not repeated herein.

It is noted that, for the multi-particulate pharmaceutical composition of the present disclosure, the respective related limitation for the first immediate-release pellet, the sustained-release pellet, the enteric-release pellet and the enteric coated sustained-release pellet mentioned above may be set independently without being limited by each other.

In one embodiment, in the multi-particulate pharmaceutical composition of the present disclosure, the mixture pellets may consist of any first immediate-release pellet mentioned above and any sustained-release pellet mentioned above. In this embodiment, the multi-particulate pharmaceutical composition of the present disclosure may have an effect of being capable of in vivo rapidly increasing the drug concentration in the blood and releasing the drug for long-term, but it is not limited thereto.

In the foregoing embodiment in which the mixture pellets of the multi-particulate pharmaceutical composition may consist of the first immediate-release pellet and the sustained-release pellet, a weight ratio of the first compound in the first immediate-release pellet to the second compound in the sustained-release pellet (ratio of drug contents in the pellets) may be about 1:0.10-10.00, such as about 1:0.25-5.00, about 1:3.5-5.5, about 1:0.1, about 1:0.5, about 1:1, about 1:2, about 1:5, about 1:10, but it is not limited thereto. In one specific embodiment, in the multi-particulate pharmaceutical composition of the present disclosure, the mixture pellets may consist of the first immediate-release pellet and the sustained-release pellet, and the weight ratio of the first compound in the first immediate-release pellet to the second compound in the sustained-release pellet may be about 1:1.

In another embodiment, in the multi-particulate pharmaceutical composition of the present disclosure, the mixture pellets may consist of any first immediate-release pellet mentioned above and any enteric-release pellet mentioned above. In this embodiment, the multi-particulate pharmaceutical composition of the present disclosure may have an effect of being capable of in vivo making the drug contained therein reach at least two peak concentrations over time to reduce the number of times the drug is taken, but it is not limited thereto.

In the foregoing embodiment in which the mixture pellets of the multi-particulate pharmaceutical composition mentioned above may consist of the first immediate-release pellet and the enteric-release pellet, the weight ratio of the first compound in the first immediate-release pellet and the third compound in the enteric-release pellet (ratio of drug contents in the pellets) may be about 1:0.10-1.00, such as about 1:0.25-0.50, about 1:3.5-5.5, about 1:0.1, about 1:0.5, about 1:1, about 1:2, about 1:5, about 1:10, etc., but it is not limited thereto. In one specific embodiment, in the multi-particulate pharmaceutical composition of the present disclosure, the mixture pellets may consist of the first immediate-release pellet and the enteric-release pellet, and the weight ratio of the first compound in the first immediate-release pellet to the third compound of the enteric-release pellet may be about 1:1.

In yet another embodiment, in the multi-particulate pharmaceutical composition of the present disclosure, the mixture pellets may consist of any sustained-release pellet mentioned above and any enteric-release pellet mentioned above. In this embodiment, the multi-particulate pharmaceutical composition of the present disclosure may have an effect of being capable of in vivo slowing down the decrease of a sustained release drug concentration in the blood during the period of rapid drug metabolism in the body to avoid premature decrease in drug concentration, but it is not limited thereto.

In the foregoing embodiment in which the mixture pellets of the multi-particulate pharmaceutical composition mentioned above may consist of the sustained-release pellet and the enteric-release pellet, a weight ratio of the second compound in the sustained-release pellet to the third compound in the enteric-release pellet (ratio of drug contents in the pellets) may be about 1:0.10-10.00, such as about 1:0.25-0.50, about 1:3.5-5.5, about 1:0.1, about 1:0.5, about 1:1, about 1:2, about 1:5, about 1:10, etc., but it is not limited thereto. In one specific embodiment, in the multi-particulate pharmaceutical composition of the present disclosure, the mixture pellets may consist of the sustained-release pellet and the enteric-release pellet, and the weight ratio of the second compound in the sustained-release pellet to the third compound in the enteric-release pellet may be about 1:1.

Also, in another embodiment, in the multi-particulate pharmaceutical composition of the present disclosure, the mixture pellets may consist of any first immediate-release pellet mentioned above, any sustained-release pellet mentioned above and any enteric-release pellet. In this embodiment, the multi-particulate pharmaceutical composition of the present disclosure can have an effect of being capable of in vivo increasing the trough concentration between at least two peak concentrations reached by the drug contained therein over time, but it is not limited thereto.

In the embodiment in which the mixture pellets of the multi-particulate pharmaceutical composition mentioned above may consist of the first immediate-release pellet, the sustained-release pellet and the enteric-release pellet, a weight ratio of the first compound in the first immediate-release pellet, the second compound in the sustained-release pellet and the third compound in the enteric-release pellet (ratio of drug contents in the pellets) may be about 1:0.10-5.00:0.10-5.00, such as about 1:0.4-0.6:3.0-5.0, about 1:3.0-5.0:3.0-5.0, about 1:0.1:0.1, about 1:0.5:0.5, about 1:1:1, about 1:2:2, about 1:5:5, about 1:10:10, about 1:0.1:10, about 1:10:0.1, about 1:2:1, about 1:1:2, about 1:5:1, about 1:1:5 etc., but it is not limited thereto. In one specific embodiment, in the multi-particulate pharmaceutical composition of the present disclosure, the mixture pellets may consist of the first immediate-release pellet, the sustained-release pellet and the enteric-release pellet, and the weight ratio of the first compound in the first immediate-release pellet, the second compound in the sustained-release pellet and the third compound in the enteric-release pellet may be about 1:1:1.

Moreover, in one embodiment, the multi-particulate pharmaceutical composition of the present disclosure, in addition to the mixture pellets mentioned above, may further comprise at least one pharmaceutically acceptable excipient, but it is not limited thereto. In this embodiment, in the multi-particulate pharmaceutical composition of the present disclosure mentioned above, a content of the mixture pellets mentioned above may be about 5-99.5 wt %, such as 5-10 wt %, 10-15 wt %, 80-90 wt %, 90-95 wt %, 5 wt %, 10 wt %, 20 wt %, 70 wt %, 80 wt %, 90 wt %, 95 wt %, 99 wt %, but it is not limited thereto. The pharmaceutically acceptable excipient mentioned above may comprise, but is not limited to, a diluent, a filler, a binder, a glidant, a disintegrant, a lubricant, etc., or any combination thereof.

The multi-particulate pharmaceutical composition of the present disclosure can be formulated into dosage forms for different administration routes utilizing conventional methods. In one embodiment, the multi-particulate pharmaceutical composition of the present disclosure mentioned above can be formulated as an oral dosage form alone or together with at least one pharmaceutically acceptable excipient. Examples of oral dosage form may comprise, capsules, pellet in capsules, granules, microgranules, tablets, coated tablets, but it is not limited thereto.

In another embodiment, the multi-particulate pharmaceutical composition of the present disclosure, in addition to the mixture pellets mentioned above, may further comprise a capsule to encapsulate the mixture pellets.

Examples of capsules may comprise, but is not limited to, hard capsules.

In addition, according to the foregoing, the present disclosure may also provide any of the immediate-release pellets mentioned above. The immediate-release pellet of the present disclosure can quickly release the drug contained therein to enable the drug quickly reach a high concentration in vivo to enable the drug work to relieve the disease and/or symptoms of a subject in need.

In addition, according to the foregoing, the present disclosure can also provide any of the aforementioned sustained-release pellets. The sustained-release pellet of the present disclosure can gradually release the drug contained therein within a period of time to make a subject in need can maintain a desired stable concentration range to enable the drug stably exert its efficacy over a long period of time to treat the disease and/or symptoms of an subject in need.

Also according to the foregoing, the present disclosure can also provide any enteric-release pellets mentioned above. The enteric-release pellet of the present disclosure can prevent the drug from being destroyed by gastric acid when moving in the digestive tract after being administered orally to a subject in need, and the drug can be effectively released and absorbed by the intestinal tract.

In addition, according to the foregoing, the present disclosure can also provide any of the enteric coated sustained-release pellets described above. The enteric coated sustained-release pellet of the present disclosure can also prevent the drug from being destroyed by gastric acid when moving in the digestive tract after being administered orally to a subject in need, and the drug can be effectively released and absorbed by the intestinal tract, and it can gradually release the drug contained therein within a period of time to enable the drug maintain a desired stable concentration range in the body of a subject in need to enable the drug can stably exert its efficacy over a long period of time to treat the disease and/or symptoms of an subject in need.

Any immediate-release pellets of the present disclosure mentioned above, any sustained-release pellets of the present disclosure mentioned above, any enteric-release pellet of the present disclosure mentioned above, or any enteric coated sustained-release pellets of the present disclosure mentioned above can also be formulated as an oral dosage form alone or together with at least one pharmaceutically acceptable excipient. Examples of oral dosage form may comprise, capsules, pellet in capsules, granules, microgranules, tablets, coated tablets, but it is not limited thereto.

Furthermore, according to the foregoing, the present disclosure can also provide use of any multi-particulate pharmaceutical composition of the present disclosure mentioned above, and the use is in the manufacture of a drug for treating autoimmune neurological diseases and/or neurodegenerative diseases. Also, in one embodiment, the multi-particulate pharmaceutical composition mentioned above may be further formulated with at least one pharmaceutically acceptable excipient into the drug for treating autoimmune neurological diseases and/or neurodegenerative diseases. The drug for treating autoimmune neurological diseases and/or neurodegenerative diseases mentioned above may be an oral dosage form. Examples of oral dosage forms, may comprise, but is not limited to, capsules, pellet in capsules, granules, microgranules, tablets and coated tablets.

Similarly, the present disclosure can also provide a use of any immediate-release pellet of the present disclosure mentioned above, any sustained-release pellet of the present disclosure mentioned above, any enteric-release pellet of the present disclosure mentioned above or any enteric coated sustained-release pellets of the present disclosure mentioned above. The use is in the manufacture of a drug for treating autoimmune neurological diseases and/or neurodegenerative diseases. In one embodiment, the immediate-release pellet of the present disclosure mentioned above, the sustained-release pellet of the present disclosure mentioned above, the enteric-release pellet of the present disclosure mentioned above or the enteric coated sustained-release pellets of the present disclosure mentioned above may be further formulated with at least one pharmaceutically acceptable excipient into the drug for treating autoimmune neurological diseases and/or neurodegenerative diseases. The drug for treating autoimmune neurological diseases and/or neurodegenerative diseases mentioned above may be an oral dosage form. Examples of oral dosage forms, may comprise, but is not limited to, capsules, pellet in capsules, granules, microgranules, tablets and coated tablets.

The relevant description of the pharmaceutically acceptable excipient mentioned in the description for any multi-particulate pharmaceutical composition of the present disclosure, any immediate-release pellet of the present disclosure mentioned above, any sustained-release pellet of the present disclosure mentioned above, any enteric-release pellet of the present disclosure mentioned above or any enteric coated sustained-release pellets of the present disclosure, or use thereof can be referred to the relevant description for the pharmaceutically acceptable excipient mentioned in the above paragraphs related to the multi-particulate pharmaceutical composition, and therefore will not be repeated here.

Furthermore, according to the foregoing, the present disclosure may also provide a method for treating autoimmune neurological diseases and/or neurodegenerative diseases. This method comprises administering an effective amount of any multi-particulate pharmaceutical composition of the present disclosure to a subject in need, but it is not limited thereto.

Furthermore, according to the foregoing, the present disclosure may also provide another method for treating autoimmune neurological diseases and/or neurodegenerative diseases. This method comprises administering an effective amount of any immediate-release pellet, any sustained-release pellet of this disclosure, any enteric-release pellet of this disclosure or any the enteric coated sustained-release pellet of this disclosure, but it is not limited thereto.

The autoimmune neurological diseases mentioned herein may comprise, multiple sclerosis (MS), neuromyelitis optica, Lambert-Eaton myasthenic syndrome, autoimmune inner ear disease, narcolepsy, neuromyotonia, Guillain-Barre syndrome, myasthenia gravis, system lupus erythematosus, transverse myelitis or acute disseminated encephalomyelitis, but it is not limited thereto. Also, examples of neurodegenerative diseases described herein may comprise, but is not limited to, Alzheimer's disease, Huntington's disease, Parkinson's disease, schizophrenia, depression, amyotrophic lateral sclerosis, multi-infarct dementia, motor neuron disease or neurofibromatosis. In one embodiment, the aforementioned autoimmune neurological diseases and/or neurodegenerative diseases mentioned above may be multiple sclerosis (MS).

The term “an effective amount” as used herein means the amount of a compound or a drug that has a therapeutic effect, or the amount of a compound or a drug that is sufficient to achieve the desired clinical improvement.

Furthermore, in any foregoing method of treating autoimmune neurological diseases and/or neurodegenerative diseases, the subject may comprise, but is not limited to, a vertebrate. Moreover, the vertebrate mentioned above may comprise a fish, an amphibian, a reptile, a bird, or a mammal, but it is not limited thereto. Examples of mammals may comprise, but are not limited to, a human, an orangutan, a monkey, a horse, a donkey, a dog, a cat, a rabbit, a guinea pig, a rat, and a mouse. In one embodiment, in any foregoing method of treating autoimmune neurological diseases and/or neurodegenerative diseases, the subject is a human.

### EXAMPLES

### Example 1: Solubility Test for Sobrerol

1. The Preparation of Dissolution Medium with Different pH Levels:

(1) Dissolution Medium with pH 2.5

7.164 g of disodium hydrogen phosphate (containing 12 molecules of water) was added to a 100 mL volumetric flask, then purified water was added to quantify to 100 mL, and then stirred using a stir bar. After the disodium hydrogen phosphate (containing 12 molecules of water) in the volumetric flask was completely dissolved, a 0.2 M disodium hydrogen phosphate solution was obtained.

19.213 g of anhydrous citric acid was assed to a 1 L volumetric flask, then purified water water was added to quantify to 1 L, and then stirred using a stir bar. After the anhydrous citric acid in the volumetric flask was completely dissolved, a 0.1 M citric acid solution was obtained.

Next, According to the list of formulas of McIlvaine buffer with different pH values, 0.2 M sodium dihydrogen phosphate solution and 0.1 M citric acid solution were mixed at a ratio of 1.71:18.29 to obtain a dissolution medium with pH 2.5.

(2) Dissolution Medium with pH 4.5

15.6 g of Sodium dihydrogen phosphate (containing 2 molecules of water) was added to a 1 L volumetric flask, then purified water water was added to quantify to 1 L, and then stirred using a stir bar. After the sodium dihydrogen phosphate (containing 2 molecules of water) in the volumetric flask was completely dissolved, a phosphate dissolution medium with pH 4.5 was obtained.

(3) Dissolution Medium with pH 6.8

7.8 g of Sodium dihydrogen phosphate (containing 2 molecules of water) and 0.94 g of sodium hydroxide were added to a 1 L volumetric flask, then purified water water was added to quantify to 1 L, and then stirred using a stir bar. After the sodium dihydrogen phosphate (containing 2 molecules of water) and sodium hydroxide in the volumetric flask were completely dissolved, a phosphate dissolution medium with pH 6.8 was obtained.

2. Solubility Test

250 mL of dissolution medium prepared above or purified water water was added to a beaker and a stir bar was added for stirring. Under stirring of the stir bar, sobrerol was slowly added to the beaker in small amounts. After each addition of sobrerol, observation was performed for 15 to 30 minutes, and then a small amount was add after confirming that sobrerol was completely dissolved. Sobrerol was continuously added until sobrerol cannot be completely dissolved within 30 minutes, and then the total amount of sobrerol added was counted, that was, the actual solubility limit of sobrerol.

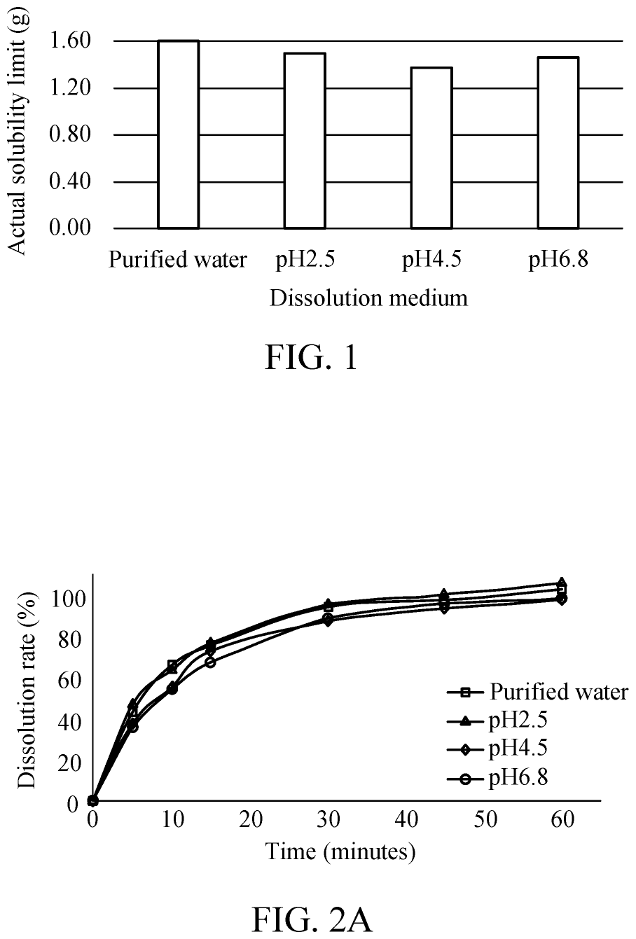

The results are shown in FIG. 1.

According to the results shown in FIG. 1, the following conclusions can be obtained: (i) The actual solubility limits of sobrerol in dissolution medium with different pH and purified water water are not much different, and thus it is confirmed that the solubility of sobrerol is not affected by pH sobrerol; (ii) The soluble amounts of sobrerol in dissolution medium with different pH and purified water water are all greater than the total amount of sobrerol taken in one day, 900 mg (based on the total amount of sobrerol taken in one day recited in The Journal of International Medical Research 1990; 18: 454-459), according to the U.S. Food and Drug Administration's guidelines “Waiver of In Vivo Bioavailability and Bioequivalence Studies for Immediate-Release Solid Oral Dosage Forms Based on a Biopharmaceutics Classification System”, sobrerol meets the specifications for high solubility drugs.

### Example 2: Preparation and Dissolution Testing of Sobrerol Immediate-Release Pellets

1. 20% Sobrerol Immediate-Release Pellets

(1) Preparation of 20% Sobrerol Immediate-Release Pellets

The immediate-release pellets containing 20% sobrerol were prepared by an Extrusion-Spheronization method. 24 g of sobrerol was mixed with 96 g of microcrystalline cellulose (the weight ratio of sobrerol to microcrystalline cellulose was 2:8), then 123.6 g of purified water water was added to perform wet granulation by a mixer. The agglomerates obtained after granulation were poured into an extruder to produce strip-shaped granules, and then the strip-shaped granules were poured into a spheronizer to produce spherical pellets. Finally, the spherical pellets were dried in an oven at 60° C. for 2-3 hours to obtain immediate-release pellets, wherein the amount of sobrerol was 20 wt %, and the immediate-release pellets were called was 20% sobrerol immediate-release pellet herein.

(2) Dissolution Testing

20% sobrerol immediate-release pellets were subjected to dissolution testing to determine the immediate-release effect thereof.

Basket method was used in the dissolution testing, and the process of the dissolution testing is described in the following.

According to the method described in Example 1, dissolution medium with different pH were prepared. 1000 mL of dissolution medium with different pH or purified water water was poured into a dissolution vessel, and then the dissolution vessel was configured with a dissolution tester for heating. When the temperature of the dissolution medium or purified water water reached 37±0.5° C., capsules containing 20% sobrerol immediate-release pellets were placed into a basket, the basket was configured with the dissolution tester with a stir bar, and then the basket was lowered into the dissolution vessel with a stirring rate of 75 rpm to begin the dissolution (initial point). Samples were taken at 5, 10, 15, 30, 45 and 60 minutes after the initial point, respectively, and sampling volume was 10 mL. The samples were filtered by a 0.45 μm filter and then component contents thereof were analyzed by high performance liquid chromatography (HPLC), respectively.

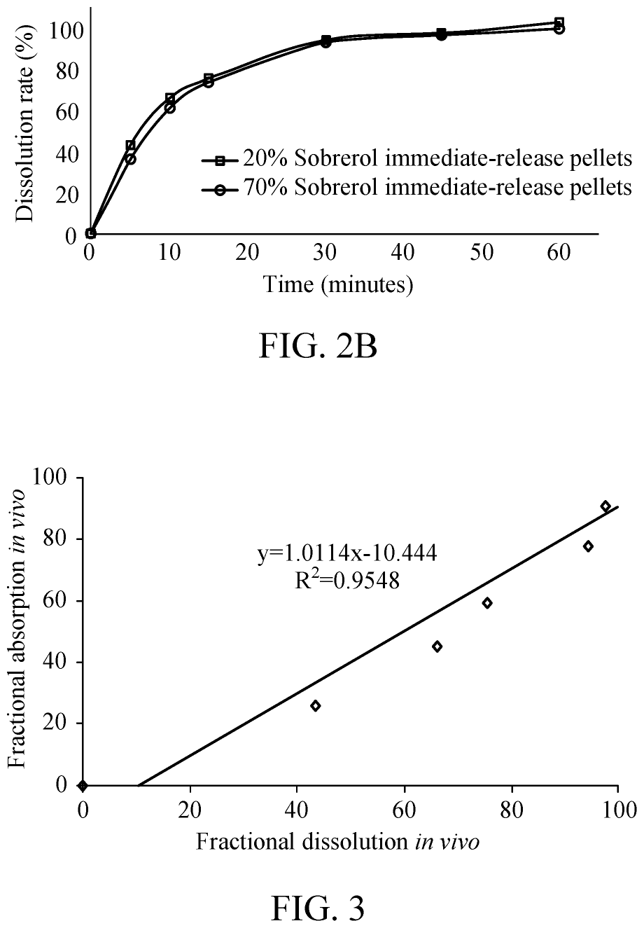

The results are shown in FIG. 2A.

According to FIG. 2A, in the dissolution tests performed by dissolution medium with different pH and performed by purified water water, the dissolution rates of the 20% sobrerol immediate-release pellets at the 30th minute after the initial point were both greater than 80%.

Moreover, according to the U.S. Food and Drug Administration' guidelines “Dissolution Testing of Immediate Release Solid Oral Dosage Forms” and the dissolution curve similarity comparison formula recited therein, the similarity (f2) comparison was performed on the dissolution curves for 20% sobrerol immediate-release pellets placed in dissolution medium with different pH and placed in purified water water (f2≥50 mean that the two dissolution curves were similar). The results are shown in Table 1.

TABLE 1
Similarity of the dissolution curve of 20% sobrerol immediate-release
pellets in dissolution medium with different pH and in purified water water
Similarity of the dissolution curve

| Groups in comparison | f2(≥50) |
|---|---|
| Purified water water and dissolution medium with | 81 |
| pH 2.5 | |
| Purified water water and dissolution medium with | 53 |
| pH 4.5 | |
| Purified water water and dissolution medium with | 58 |
| pH 6.8 | |

Based on the guidelines of the US Food and Drug Administration “Dissolution Testing of Immediate Release Solid Oral Dosage Forms” and Table 1, it is known that, the dissolution curves of 20% sobrerol immediate-release pellets in dissolution medium with different pH and in purified water water are similar (f2≥50), which means that the solubility of sobrerol after wet granulation is not affected by pH.

2. 70% Sobrerol Immediate-Release Pellets

(1) Preparation of 70% Sobrerol Immediate-Release Pellet

The immediate-release pellets containing 70% sobrerol were prepared by an extrusion and rounding method. 84 g of sobrerol was mixed with 36 g of microcrystalline cellulose (the weight ratio of sobrerol to microcrystalline cellulose was 7:3), then 72 g of purified water water was added to perform wet granulation by a mixer. The agglomerates obtained after granulation were poured into an extruder to produce strip-shaped pellets, and then the strip-shaped pellets were poured into a rounding machine to produce spherical pellets. Finally, the spherical pellets were dried in an oven at 60° C. for 2-3 hours to obtain immediate-release pellets, wherein the amount of sobrerol was 70 wt %, and the immediate-release pellets were called was 20% sobrerol immediate-release pellet herein.

(2) Dissolution Testing

70% sobrerol immediate-release pellets were subjected to dissolution testing to determine the immediate-release effect thereof.

Basket method was used in the dissolution testing, and the process of the dissolution testing is described in the following.

1000 mL of purified water water was poured into a dissolution vessel, and then the dissolution vessel was configured with a dissolution tester for heating. When the temperature of the purified water water reached 37±0.5° C., capsules containing 70% sobrerol immediate-release pellets were placed into a basket, the basket was configured with the dissolution tester with a stir bar, and then the basket was lowered into the dissolution vessel with a stirring rate of 75 rpm to begin the dissolution (initial point). Samples were taken at 5, 10, 15, 30, 45 and 60 minutes after the initial point, respectively, and sampling volume was 10 mL. The samples were filtered by a 0.45 μm filter and then component contents thereof were analyzed by high performance liquid chromatography (HPLC), respectively.

The results are shown in FIG. 2B.

According to FIG. 2B, in the dissolution tests performed by purified water water, the dissolution rates of the 70% sobrerol immediate-release pellets at the 30th minute after the initial point was greater than 80%.

Furthermore, according to the U.S. Food and Drug Administration' guidelines “Dissolution Testing of Immediate Release Solid Oral Dosage Forms” and the dissolution curve similarity comparison formula recited therein, the similarity (f2) comparison was performed on the dissolution curve for 20% sobrerol immediate-release pellets placed in purified water water and the dissolution curve for 70% sobrerol immediate-release pellets placed in purified water water (f2≥50 mean that the two dissolution curves were similar).

The results are shown in Table 2.

TABLE 2
Similarity of the dissolution curves of 20% sobrerol immediate-release
pellet and 70% sobrerol immediate-release pellet in purified water water
Similarity of the dissolution curve

| Groups in comparison | f2(≥50) |
|---|---|
| 20% sobrerol immediate-release pellets and | 66 |
| 70% sobrerol immediate-release pellets | |

Based on the guidelines of the US Food and Drug Administration “Dissolution Testing of Immediate Release Solid Oral Dosage Forms” and Table 2, it is known that the immediate-release pellets with different contents of sobrerol have similar dissolution curves in purified water water, and this verifies extrusion-rounding method can be used to prepare immediate-release pellets with a sobrerol content of 20-70 wt %.

### Example 3: Evaluation of the Correlation Between In Vitro Dissolution and In Vivo Dissolution (In Vivo Absorption) of Sobrerol Immediate-Release Pellets

In the literature, Eur J Clin Pharmacol (1983) 24:209-215, is a study of the dynamic performance of the active ingredient sobrerol in the human body, and in this literature, a clinical trial result on the blood concentration in humans after oral administration of 300 mg of sobrerol is shown.

Using the two-compartment model of mass balance methods, the clinical trial result on the blood concentration in humans after oral administration of 300 mg of sobrerol (Eur J Clin Pharmacol (1983) 24:209-215) was converted to in vivo absorption rate (also known as in vivo dissolution) to obtain an in vivo absorption graph, and according to the method described in International Journal of Pharmaceutics 418 (2011) 142-148, the above-mentioned sobrerol in vivo absorption graph was calculated by deconvolution to obtain the in vivo absorption percentage curve.

This in vivo absorption percentage curve was compared with the dissolution curve of 20% sobrerol immediate-release pellets in purified water water, and the linear relationship between the two was calculated.

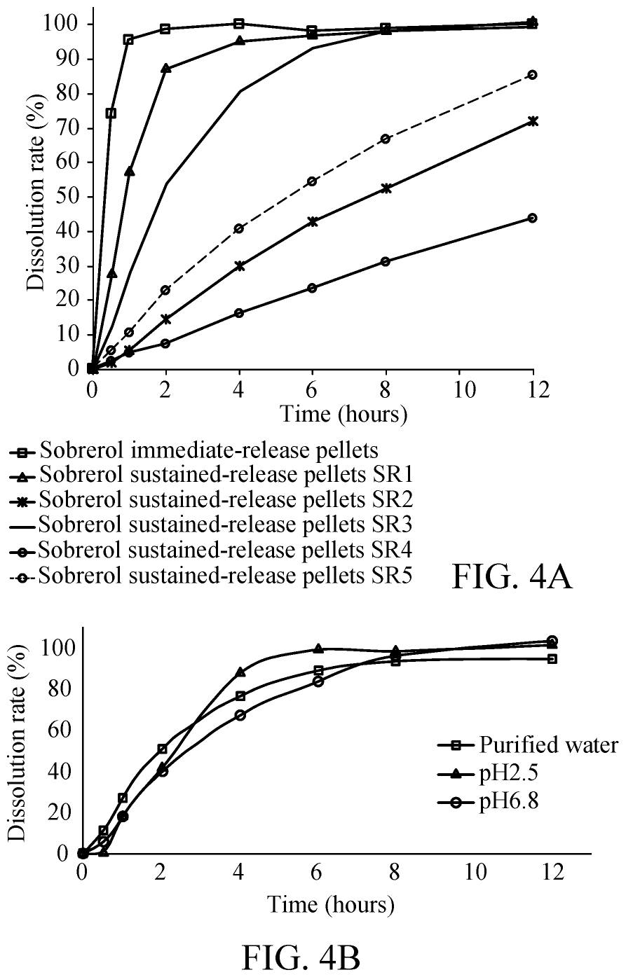

The results are shown in FIG. 3.

Based on FIG. 3, it is known that the R-squared value of the two curves is 0.9548, and this shows that the two curves have a good linear relationship, verifying that the in vitro dissolution and in vivo dissolution of sobrerol have a highly positive correlation.

Therefore, based on the in vitro dissolution result of sobrerol immediate-release pellets, in vivo absorption of sobrerol can be evaluated.

### Example 4: Preparation and Dissolution Testing of Sustained-Release Pellets

1. Preparation of Sobrerol Sustained-Release Pellets

According to the different formulations shown in Table 3, a spraying solution for forming sustained-release layer was prepared. 180 g of 20% sobrerol immediate-release pellets (particle size about 600-1000 μm) was introduced into a fluid-bed granulator and spraying-coated with the spraying solution (spray volume: 0.6-1.8 g/minute; spray air pressure: 1.0-1.4 kg/cm²; exhaust air temperature: 20-25° C.; inlet air temperature: 25-35° C.). After the spraying solution is sprayed, pellets coated with the spraying solution were taken out and placed in an oven, and a 2-hour curing process at different curing temperatures based on the formulas shown in Table 3 was performed thereon to obtain the sobrerol sustained-release pellets.

TABLE 3
Formulas of sustained-release pellets with
different the sustained-release layers

| | Sobrerol sustained-release pellets | | | | |
|---|---|---|---|---|---|
| | SR1 | SR2 | SR3 | SR4 | SR5 |
| Ingredient | Usage amount (g) | | | | |
| 20% sobrerol immediate- | 180 | 180 | 180 | 180 | 180 |
| release pellets | | | | | |
| Ethyl cellulose | 11.54 | 14.00 | 9.62 | 10.42 | 10.00 |
| Hydroxypropyl methyl | 3.46 | 3.50 | 2.88 | 2.08 | 2.50 |
| cellulose | | | | | |
| Ethanol | 115.40 | 140.00 | 96.20 | 104.20 | 100.00 |
| Purified water water | 34.60 | 35.00 | 28.80 | 20.80 | 25.00 |
| Curing temperature | 70° C. | 70° C. | 60° C. | 60° C. | 50° C. |

2. Dissolution Testing

(1) Dissolution Testing in Purified Water Water

The obtained sustained-release pellets mentioned above were subjected to dissolution testing to determine the sustained-release effect thereof.

1000 mL of purified water water was poured into a dissolution vessel, and then the dissolution vessel was configured with a dissolution tester for heating. When the temperature of the purified water water reached 37±0.5° C., capsules containing sustained-release pellets were placed into a basket, the basket was configured with the dissolution tester with a stir bar, and then the basket was lowered into the dissolution vessel with a stirring rate of 75 rpm to begin the dissolution (initial point). Samples were taken at 0.5, 1, 2, 4, 6, 8 and 12 hours after the initial point, respectively, and sampling volume was 10 mL. The samples were filtered by a 0.45 μm filter and then component contents thereof were analyzed by high performance liquid chromatography (HPLC), respectively.

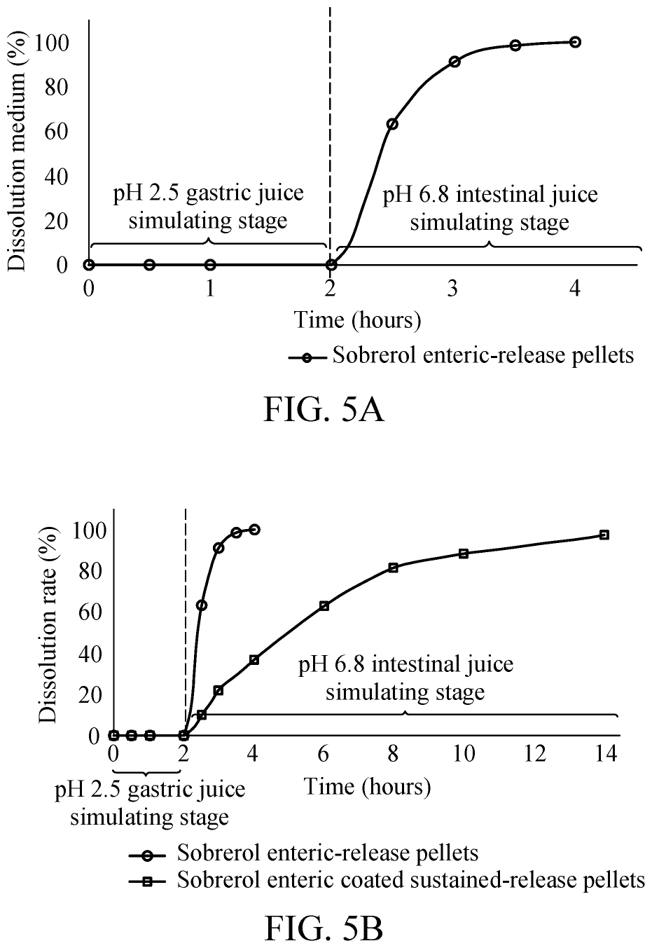

The results are shown in FIG. 4A.

According to FIG. 4A, it can be known that the sustained-release pellets with different sustained-release layers formed by different formula have different dissolution curves, and the dissolution rates reach about 70%-100% within 6-12 hours, wherein only the dissolution rate of SR5 at the 12^{th} hour is less than 70%. This result shows that the sustained-release layers formed by different formulas all have the effects of delayed release, wherein the sustained-release pellets SR3 have a dissolution rate greater than about 90% at the 6^{th} hour, and a dissolution rate close to 100% at the 8^{th} hour, and they have excellent sustained release performance, and can be used as a representative of the sustained-release pellets in a multi-particulate system.

(2) Dissolution Testing in Dissolution Medium with Different pH

The sustained-release pellet SR3 was subjected to a dissolution testing in dissolution medium with different pH levels to determine whether the sustained-release layer was affected by pH.

Basket method was used in the dissolution testing, and the process of the dissolution testing is described in the following.

According to the method described in Example 1, dissolution medium with different pH were prepared. 1000 mL of dissolution medium with different pH was poured into a dissolution vessel, and then the dissolution vessel was configured with a dissolution tester for heating. When the temperature of the dissolution medium or purified water water reached 37±0.5° C., capsules containing the sustained-release pellets SR3 were placed into a basket, the basket was configured with the dissolution tester with a stir bar, and then the basket was lowered into the dissolution vessel with a stirring rate of 75 rpm to begin the dissolution (initial point). Samples were taken at 0.5, 1, 2, 4, 6, 8 and 12 hours after the initial point, respectively, and sampling volume was 10 mL. The samples were filtered by a 0.45 μm filter and then component contents thereof were analyzed by high performance liquid chromatography (HPLC), respectively.

The results are shown in FIG. 4B.

According to FIG. 4B, it can be known that for sustained-release pellets SR3 in dissolution medium with different pH, the dissolution rates at the 6th hour all are greater than 80%, and the dissolution rates at the 8th hour are close to 100%. This result indicates that the difference in pH will not significantly affect the effect of the sustained-release layer.

### Example 5: Preparation and Dissolution Testing of Enteric-Release Pellets

1. Preparation of Sobrerol Enteric-Release Pellets

140.00 g of Eudragit L30D-55 (containing methacrylic acid-ethyl acrylate copolymer), 35.70 g of PlasAcryl HTP20 (containing glyceryl monostearate, polysorbate 80, triethyl citrate and water) and 70.00 g of purified water water were placed into a beaker and stirred with a paddle mixer for 10 minutes to form a suspend dispersion solution. The suspension dispersion solution was filtered through a 30 mesh sieve to obtain a spraying solution for forming the enteric-release layer. 200 g of 20% sobrerol immediate-release pellets (particle size: about 600-1000 μm) was introduced into a fluid-bed granulator and spraying-coated with the spraying solution (spray volume: 0.8-2.0 g/minute; spray air pressure: 1.0-1.2 kg/cm²; exhaust air temperature: 25-35° C.; inlet air temperature: 44-45° C.) to obtain sobrerol the enteric-release pellets.

2. Dissolution Testing

The obtained sobrerol enteric-release pellets mentioned above were subjected to dissolution testing to determine the enteric-release effect thereof.

Basket method was used in the dissolution testing, and the process of the dissolution testing is described in the following.

According to the method described in Example 1, dissolution medium with pH 2.5 and pH 6.8 were prepared. 1000 mL of dissolution medium with pH 2.5 and 1000 mL of dissolution medium with pH 6.8 were respectively poured into a dissolution vessel, and then the dissolution vessel was configured with a dissolution tester for heating. When the temperature of the dissolution medium or purified water water reached 37±0.5° C., capsules containing sobrerol enteric-release pellets were placed into a basket, the basket was configured with the dissolution tester with a stir bar, and then the basket was lowered into the dissolution vessel containing 1000 mL of dissolution medium with pH 2.5 with a stirring rate of 75 rpm to begin the dissolution (first initial point). Samples were taken at 0.5, 1 and 2 hours after the first initial point, respectively, and sampling volume was 10 mL. After the sampling at the 2^{nd} hour mentioned above, the basket containing sobrerol enteric-release pellets was immediately taken out and then placed into the dissolution vessel containing 1000 mL of dissolution medium with pH 6.8 with a stirring rate of 75 rpm to begin the intestinal juice simulated dissolution (second initial point). Samples were taken at 0.5, 1, 1.5 and 2 hours after the second initial point, respectively, and sampling volume was 10 mL. The samples were filtered by a 0.45 μm filter and then component contents thereof were analyzed by high performance liquid chromatography (HPLC), respectively.

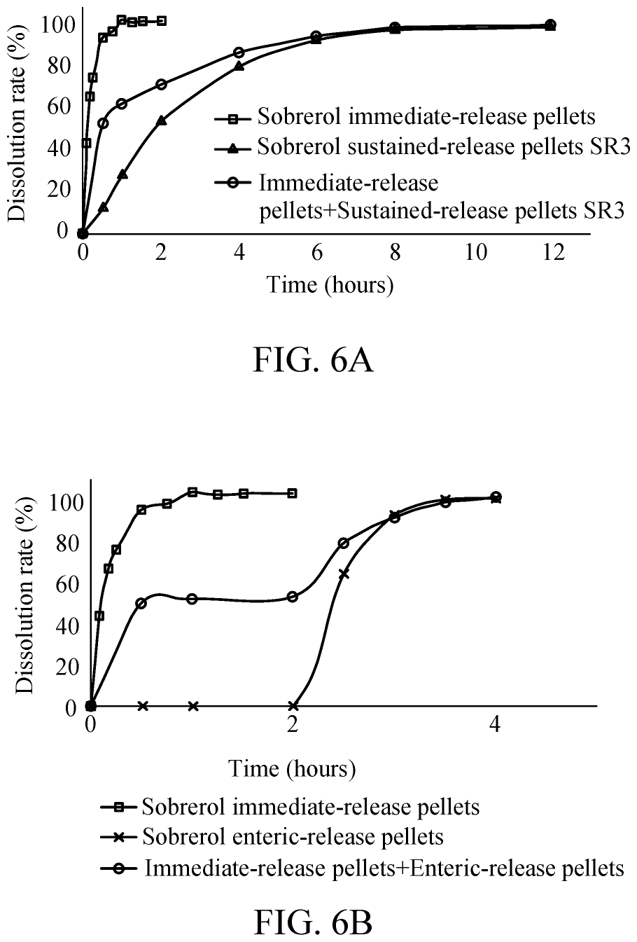

The results are shown in FIG. 5A.

According to FIG. 5A, it can be known that the dissolution rate of sobrerol enteric-release pellets in the dissolution medium with pH 2.5 which simulates gastric juice is 0, which means that sobrerol is not released during the pH 2.5 gastric juice simulating stage, and the coating of the enteric-release layer is intact. At the 1st hour after the sobrerol enteric-release pellets are transferred from the dissolution medium with pH 2.5 which simulates gastric juice to the dissolution medium with pH 6.8 which simulates intestinal juice, the dissolution rate is greater than 80%, which means that the enteric-release layer of the sobrerol enteric-release pellets during the pH 6.8 intestinal juice simulating stage indeed dissolve, and the drug contained in the sobrerol enteric-release pellets will be dissolved out.

### Example 6: Preparation and Dissolution Testing of the Enteric Coated Sustained-Release Pellet

Enteric-release pellets with sustained release effects belong to a kind of enteric-release pellets although not all drugs and excipients are suitable for the preparation of such particles. The purpose of this part of the test is to confirm that the formula used in the present disclosure is suitable for the preparation of the enteric coated sustained-release pellets, and enteric-release pellets with sustained release effect can be indeed obtained thereby.

1. Preparation of Sobrerol Enteric Coated Sustained-Release Pellets

115.50 g of Eudragit L30D-55, 29.45 g of PlasAcryl HTP20 and 57.75 g of purified water water were placed into a beaker and, stirred with a paddle mixer for 10 minutes to form a suspension dispersion solution. The suspension dispersion solution was filtered through a 30 mesh sieve to obtain a spraying solution for forming the enteric-release layer. 165 g of the sustained-release pellets SR3 (particle size about 600-1050 μm) was introduced into a fluid-bed granulator and spraying-coated with the spraying solution (spray volume: 0.8-2.0 g/minute; spray air pressure: 1.0-1.2 kg/cm²; exhaust air temperature: 25-35° C.; inlet air temperature: 44-45° C.) to obtain sobrerol enteric coated sustained-release pellets.

2. Dissolution Testing

Basket method was used in the dissolution testing, and the process of the dissolution testing is described in the following.

According to the method described in Example 1, dissolution medium with pH 2.5 and pH 6.8 were prepared. 1000 mL of dissolution medium with pH 2.5 and 1000 mL of dissolution medium with pH 6.8 were respectively poured into a dissolution vessel, and then the dissolution vessel was configured with a dissolution tester for heating. When the temperature of the dissolution medium reached 37±0.5° C., capsules containing sobrerol enteric coated sustained-release pellets were placed into a basket, the basket was configured with the dissolution tester with a stir bar, and then the basket was lowered into the dissolution vessel containing 1000 mL of dissolution medium with pH 2.5 with a stirring rate of 75 rpm to begin the dissolution (first initial point). Samples were taken at 0.5, 1 and 2 hours after the first initial point, respectively, and sampling volume was 10 mL. After the sampling at the 2^{nd} hour mentioned above, the basket containing sobrerol enteric-release pellets was immediately taken out and then placed into the dissolution vessel containing 1000 mL of dissolution medium with pH 6.8 with a stirring rate of 75 rpm to begin the intestinal juice simulated dissolution (second initial point). Samples were taken at 0.5, 1, 2, 4, 6, 8 and 12 hours after the second initial point, respectively, and sampling volume was 10 mL. The samples were filtered by a 0.45 μm filter and then component contents thereof were analyzed by high performance liquid chromatography (HPLC), respectively.

The results are shown in FIG. 5B.

According to FIG. 5B, it can be known that the dissolution rate of sobrerol enteric coated sustained-release pellets in the dissolution medium with pH 2.5 which simulates gastric juice is 0, which means that sobrerol is not released during the pH 2.5 gastric juice simulating stage, and the coating of the enteric-release layer is intact. At the 6f hour after the sobrerol enteric coated sustained-release pellets are transferred from the dissolution medium with pH 2.5 which simulates gastric juice to the dissolution medium with pH 6.8 which simulates intestinal juice, the dissolution rate is greater than 80%, which means that the enteric-release layer of the sobrerol enteric-release pellets during the pH 6.8 intestinal juice simulating stage and the sustained release effect of the sobrerol enteric coated sustained-release pellets is similar to that of the sustained-release pellets SR3 in Example 4.

### Example 6: Dissolution Testing of Multi-Particulate

1. Multi-particulate: a combination of immediate-release pellets and sustained-release pellets (the weight ratio of sobrerol in immediate-release pellets and sobrerol in sustained-release pellets was 1:1)

Dissolution Testing

The 20% sobrerol immediate-release pellets prepared in the Example mentioned above was mixed with the sobrerol sustained-release pellets SR3 to form the mixture pellets (the weight ratio of sobrerol in immediate-release pellets and sobrerol in sustained-release pellets was 1:1), and the mixture pellets were subjected to dissolution testing to determine the effect thereof.

Basket method was used in the dissolution testing, and the process of the dissolution testing is described in the following.

1000 mL of purified water water was poured into a dissolution vessel, and then the dissolution vessel was configured with a dissolution tester for heating. When the temperature of the purified water water reached 37±0.5° C., capsules containing 20% sobrerol immediate-release pellets were placed into a basket, the basket was configured with the dissolution tester with a stir bar, and then the basket was lowered into the dissolution vessel with a stirring rate of 75 rpm to begin the dissolution (initial point). Samples were taken at 5, 10, 15, 30, 45, 60, 75, 90 and 120 minutes after the initial point, respectively, and sampling volume was 10 mL. The samples were filtered by a 0.45 μm filter and then component contents thereof were analyzed by high performance liquid chromatography (HPLC), respectively.

In addition, 1000 mL of purified water water was poured into a dissolution vessel, and then the dissolution vessel was configured with a dissolution tester for heating. When the temperature of the purified water water reached 37±0.5° C., capsules containing 20% sobrerol sustained-release pellets SR3 or the mixture pellets mentioned above were placed into a basket, the basket was configured with the dissolution tester with a stir bar, and then the basket was lowered into the dissolution vessel with a stirring rate of 75 rpm to begin the dissolution (initial point). Samples were taken at 0.5, 1, 2, 4, 6, 8 and 12 hours after the initial point, respectively, and sampling volume was 10 mL. The samples were filtered by a 0.45 μm filter and then component contents thereof were analyzed by high performance liquid chromatography (HPLC), respectively.

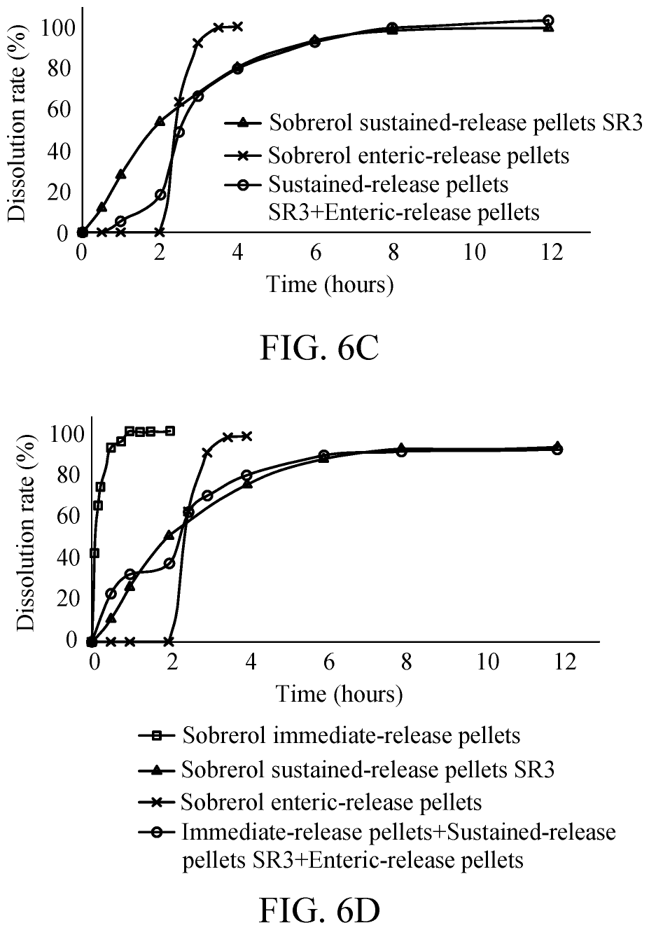

The results are shown in FIG. 6A.

According to FIG. 6A, it can be known that a dissolution rate of the sobrerol immediate-release pellets alone reaches 100% at the 1^{st} hour during the dissolution testing, and this can be regarded as 100% release within 1 hour after taking the drug in a human trial, and then sobrerol should be metabolized in the human body, and thus the time can maintain the drug concentration may be shorter. In the first 2 hours of the dissolution test, the dissolution rate of the sobrerol sustained-release pellets alone is less than 60%, and the dissolution rate is about complete release at the 8^{th} hour, and this can be regarded as a slow drug release curve in a human trial, and there should be a certain drug concentration within 8 hours after taking the drug, but the drug concentration may be low in the first 2 hours after taking the drug and cannot produce a certain medicinal effect.

In contrast, the dissolution curve of multi-particulate has approximately half the dissolution efficiency of the sobrerol immediate-release pellets and half the dissolution efficiency of the sobrerol sustained-release pellets. Compared to the presence of the sobrerol immediate-release pellets alone, the multi-particulate can increase the dissolution rate in the first 2 hours of the dissolution test, and compared to the presence of the sobrerol immediate-release pellets alone, the multi-particulate can extend time for reaching 100% dissolution in the dissolution test. This can be regarded as a rapid increase to 60% of the drug release in the first 2 hours after taking multiple particles in a human trial. Compared to the sobrerol sustained-release pellets, the multi-particulate can start the drug effect earlier, and the drug release thereof can be extended to 8 hours, and compared to the sobrerol immediate-release pellets, the multi-particulate can have a drug concentration in the body for longer time and prolong the medicinal effect without the need to take the drug again in a short time, and can have a longer drug taking interval.

2. Multi-particulate: a combination of immediate-release pellets and enteric-release pellets (the weight ratio of sobrerol in immediate-release pellets and sobrerol in enteric-release pellets was 1:1)

Dissolution Testing

The 20% sobrerol immediate-release pellets prepared in the Example mentioned above was mixed with the sobrerol enteric-release pellets to form the mixture pellets (the weight ratio of sobrerol in immediate-release pellets and sobrerol in enteric-release pellets was 1:1), and the mixture pellets were subjected to dissolution testing to determine the effect thereof.

Basket method was used in the dissolution testing, and the process of the dissolution testing is described in the following.

1000 mL of purified water water was poured into a dissolution vessel, and then the dissolution vessel was configured with a dissolution tester for heating. When the temperature of the purified water water reached 37±0.5° C., capsules containing 20% sobrerol immediate-release pellets were placed into a basket, the basket was configured with the dissolution tester with a stir bar, and then the basket was lowered into the dissolution vessel with a stirring rate of 75 rpm to begin the dissolution (initial point). Samples were taken at 5, 10, 15, 30, 45, 60, 75, 90 and 120 minutes after the initial point, respectively, and sampling volume was 10 mL. The samples were filtered by a 0.45 μm filter and then component contents thereof were analyzed by high performance liquid chromatography (HPLC), respectively.

Moreover, according to the method described in Example 1, dissolution medium with pH 2.5 and pH 6.8 were prepared. 1000 mL of dissolution medium with pH 2.5 and 1000 mL of dissolution medium with pH 6.8 were respectively poured into a dissolution vessel, and then the dissolution vessel was configured with a dissolution tester for heating. When the temperature of the dissolution medium reached 37±0.5° C., capsules containing sobrerol enteric-release pellets or the mixture pellets mentioned above were placed into a basket, the basket was configured with the dissolution tester with a stir bar, and then the basket was lowered into the dissolution vessel containing 1000 mL of dissolution medium with pH 2.5 with a stirring rate of 75 rpm to begin the dissolution (first initial point). Samples were taken at 0.5, 1 and 2 hours after the first initial point, respectively, and sampling volume was 10 mL. After the sampling at the 2^{nd} hour mentioned above, the basket containing the sobrerol enteric-release pellets or the mixture pellets mentioned above was immediately taken out and then placed into the dissolution vessel containing 1000 mL of dissolution medium with pH 6.8 with a stirring rate of 75 rpm to begin the intestinal juice simulated dissolution (second initial point). Samples were taken at 0.5, 1, 1.5 and 2 hours after the second initial point, respectively, and sampling volume was 10 mL. The samples were filtered by a 0.45 μm filter and then component contents thereof were analyzed by high performance liquid chromatography (HPLC), respectively.

The results are shown in FIG. 6B.

According to FIG. 6B, it can be known that a dissolution rate of the sobrerol immediate-release pellets alone reaches 100% at the 1^{st} hour during the dissolution testing, and this can be regarded as 100% release within 1 hour after taking the drug in a human trial, and then sobrerol should be metabolized in the human body, and thus the time can maintain the drug concentration may be shorter. The sobrerol enteric-release pellets alone only can be dissolved the drug out in the intestinal juice simulating stage, and reach 100% dissolution at 1.5 hours in the intestinal juice simulating stage, and this can be regarded as in a human trial, after taking the medicine, there will be no release of the medicine before it reaches the small intestine, but it will be released quickly after it reaches the small intestine.

The dissolution curve of the multi-particulate system composed of the sobrerol immediate-release pellets and the sobrerol enteric-release pellets has approximately half the dissolution efficiency of the sobrerol immediate-release pellets and half the dissolution efficiency of the sobrerol enteric release, shows the dissolution efficacy of the sobrerol immediate-release pellets at the gastric juice simulating stage and the dissolution efficacy of the sobrerol enteric-release pellets at the intestinal juice simulating stage, and the two together form two dissolution curve fluctuations. This can be regarded as a pulse of drug concentration in a human trial, that is, the drug concentration curve will appear twice the highest drug concentration in the blood, which means double peaks, and this is similar to the peaks formed by taking the medicine twice, and thus it has the benefit of once less for taking medicine.

3. Multi-particulate: a combination of sustained-release pellets and enteric-release pellets (the weight ratio of sobrerol in sustained-release pellets and sobrerol in enteric-release pellets was 1:1).

Dissolution Testing

The sustained-release pellets SR3 prepared and the sobrerol enteric-release pellets prepared in the Examples mentioned above were mixed to form the mixture pellets (the weight ratio of sobrerol in sustained-release pellets and sobrerol in enteric-release pellets was 1:1), and the mixture pellets were subjected to dissolution testing to determine the effect thereof.

Basket method was used in the dissolution testing, and the process of the dissolution testing is described in the following.

1000 mL of purified water water was poured into a dissolution vessel, and then the dissolution vessel was configured with a dissolution tester for heating. When the temperature of the purified water water reached 37±0.5° C., capsules containing the sustained-release pellets SR3 were placed into a basket, the basket was configured with the dissolution tester with a stir bar, and then the basket was lowered into the dissolution vessel with a stirring rate of 75 rpm to begin the dissolution (initial point). Samples were taken at 0.5, 1, 2, 4, 6, 8 and 12 hours after the initial point, respectively, and sampling volume was 10 mL. The samples were filtered by a 0.45 μm filter and then component contents thereof were analyzed by high performance liquid chromatography (HPLC), respectively.

According to the method described in Example 1, dissolution medium with pH 2.5 and pH 6.8 were prepared. 1000 mL of dissolution medium with pH 2.5 and 1000 mL of dissolution medium with pH 6.8 were respectively poured into a dissolution vessel, and then the dissolution vessel was configured with a dissolution tester for heating. When the temperature of the dissolution medium reached 37±0.5° C., capsules containing the enteric-release pellets or the mixture pellets mentioned above were placed into a basket, the basket was configured with the dissolution tester with a stir bar, and then the basket was lowered into the dissolution vessel containing 1000 mL of dissolution medium with pH 2.5 with a stirring rate of 75 rpm to begin the dissolution (first initial point). Samples were taken at 0.5, 1 and 2 hours after the first initial point, respectively, and sampling volume was 10 mL. After the sampling at the 2^{nd} hour mentioned above, the basket containing the enteric-release pellets or the mixture pellets mentioned above were immediately taken out and then placed into the dissolution vessel containing 1000 mL of dissolution medium with pH 6.8 with a stirring rate of 75 rpm to begin the intestinal juice simulated dissolution (second initial point). Samples were taken at 0.5, 1, 1.5 and 2 hours (for the enteric-release pellets) or at 0.5, 1, 2, 4, 6 and 10 hours (for the mixture pellets of the sustained-release pellets and the enteric-release pellets) after the second initial point, respectively, and sampling volume was 10 mL. The samples were filtered by a 0.45 μm filter and then component contents thereof were analyzed by high performance liquid chromatography (HPLC), respectively.

The results are shown in FIG. 6C.

According to FIG. 6C, it can be known that the dissolution curve of the multi-particulate system composed of the sobrerol immediate-release pellets and the sobrerol enteric-release pellets has approximately half the dissolution efficiency of the sobrerol sustained-release pellets and half the dissolution efficiency of the sobrerol enteric release. Therefore, compared to the sustained-release pellets alone, the dissolution rate of the multi-particulate in the gastric juice simulating stage is lower, but the dissolution rate of the multi-particulate will be increased when it is transferred to in the intestinal juice simulating stage due to the effect of the enteric-release pellets, and the overall drug release time is at least up to 8 hours.

The design purpose of the multi-particulate composed of sobrerol sustained-release pellets and sobrerol the enteric-release pellets is: (i) After the human body takes the drug, drug metabolism occurs with the release and absorption of the drug. When the drug metabolism is strong, it may cause a drug concentration of a sustained release drug with a certain release rate to be a plain shape in the human body, and it turns into a gentle slope-like drug concentration which decreases earlier, and the efficacy of the drug may be low at this time. However, with the rapid release of sobrerol the enteric-release pellets in the middle section of daily time, the multi-particulate may be able to offset certain drug metabolism and ensure long-term drug efficacy; (ii) With daily eating, chyme will be produced in the gastrointestinal tract. This chyme has a certain chance to wrap the sustained-release pellets and block the release of the drug from the water-soluble holes of the sustained-release layer. However, combining the use of enteric-release pellets whose enteric layer will collapse as a whole can support the drug release of at this stage and avoid premature drop in drug concentration.

4. Multi-particulate: a combination of immediate-release pellets, sustained-release pellets and enteric-release pellets (the weight ratio of sobrerol in immediate-release pellets, sobrerol in sustained-release pellets and sobrerol in enteric-release pellets was 1:1:1).

Dissolution Testing

The 20% sobrerol immediate-release pellets, the sustained-release pellets SR3 prepared and the sobrerol enteric-release pellets prepared in the Examples mentioned above were mixed to form the mixture pellets (the weight ratio of sobrerol in immediate-release pellets, sobrerol in sustained-release pellets and sobrerol in enteric-release pellets was 1:1:1), and the mixture pellets were subjected to dissolution testing to determine the effect thereof.

Basket method was used in the dissolution testing, and the process of the dissolution testing is described in the following.

1000 mL of purified water water was poured into a dissolution vessel, and then the dissolution vessel was configured with a dissolution tester for heating. When the temperature of the purified water water reached 37±0.5° C., capsules containing the 20% sobrerol immediate-release pellets or the sustained-release pellets SR3 were placed into a basket, the basket was configured with the dissolution tester with a stir bar, and then the basket was lowered into the dissolution vessel with a stirring rate of 75 rpm to begin the dissolution (initial point). Samples were taken at 5, 10, 15, 30, 45, 60, 75, 90 and 120 minutes (for the immediate-release pellets) or at 0.5, 1, 2, 4, 6, 8 and 12 hours (for the sustained-release pellets SR3) after the initial point, respectively, and sampling volume was 10 mL. The samples were filtered by a 0.45 μm filter and then component contents thereof were analyzed by high performance liquid chromatography (HPLC), respectively.

According to the method described in Example 1, dissolution medium with pH 2.5 and pH 6.8 were prepared. 1000 mL of dissolution medium with pH 2.5 and 1000 mL of dissolution medium with pH 6.8 were respectively poured into a dissolution vessel, and then the dissolution vessel was configured with a dissolution tester for heating. When the temperature of the dissolution medium reached 37±0.5° C., capsules containing the enteric-release pellets or the mixture pellets mentioned above were placed into a basket, the basket was configured with the dissolution tester with a stir bar, and then the basket was lowered into the dissolution vessel containing 1000 mL of dissolution medium with pH 2.5 with a stirring rate of 75 rpm to begin the dissolution (first initial point). Samples were taken at 0.5, 1 and 2 hours after the first initial point, respectively, and sampling volume was 10 mL. After the sampling at the 2^{nd} hour mentioned above, the basket containing the enteric-release pellets or the mixture pellets mentioned above were immediately taken out and then placed into the dissolution vessel containing 1000 mL of dissolution medium with pH 6.8 with a stirring rate of 75 rpm to begin the intestinal juice simulated dissolution (second initial point). Samples were taken at 0.5, 1, 1.5 and 2 hours (for the enteric-release pellets) or at 0.5, 1, 2, 4, 6 and 10 hours (for the mixture pellets of the immediate-release pellets, the sustained-release pellets and the enteric-release pellets) after the second initial point, respectively, and sampling volume was 10 mL. The samples were filtered by a 0.45 μm filter and then component contents thereof were analyzed by high performance liquid chromatography (HPLC), respectively.

The results are shown in FIG. 6D.

According to FIG. 6D, it can be known that the dissolution curve of the multi-particulate composed of the sobrerol immediate-release pellets, the sobrerol immediate-release pellets and the sobrerol enteric-release pellets has the same efficacy as the immediate-release pellets, the sobrerol sustained-release pellets and the sobrerol enteric-release pellets at the same time. Compared to the sobrerol immediate-release pellets, the multi-particulate can have a longer drug release time. Compared to the sobrerol sustained-release pellets, the multi-particulate can have an earlier drug release and a higher drug release concentration than the sobrerol. Compared to the sobrerol the enteric-release pellets, the multi-particulate can have a drug release concentration before the drug reaches the small intestine.

Compared to the combination of the immediate-release pellets and the enteric-release pellets, the multi-particulate composed of the sobrerol immediate-release pellets, the sobrerol immediate-release pellets and the sobrerol enteric-release pellets have the sustained-release pellets which can be used to connect two peaks to avoid that the trough between the peaks of the 2 peaks is too low to cause the interruption of the drug effect which is not conducive to the treatment of chronic diseases, etc.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A multi-particulate pharmaceutical composition, comprising:
mixture pellets, which comprise at least two of the following pellets:
a first immediate-release pellet, a first sustained-release pellet, an enteric-release pellet and an enteric coated sustained-release pellet,
wherein the first immediate-release pellet is composed of a first medicated mixture, and the first medicated mixture comprises:
a first compound, or its geometric isomer, enantiomer, diastereomer or pharmaceutically acceptable salt; and
a first excipient composition,
wherein a content of the first compound in the first medicated mixture is 5-85 wt %, and the first compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 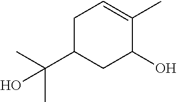 |
| Formula (II) | 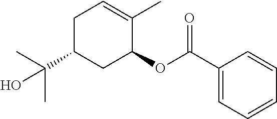 [SMILES: CC1=CC[C@@H](C(C)(C)O)C[C@@H]1OC(=O)c1ccccc1] |
| Formula (III) | 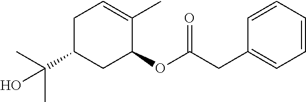 [SMILES: CC1=CC[C@@H](C(C)(C)O)C[C@@H]1OC(=O)Cc1ccccc1] |
| Formula (IV) | 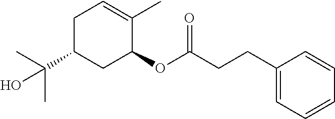 [SMILES: CC1=CC[C@@H](C(C)(C)O)C[C@@H]1OC(=O)CCc1ccccc1] |
| Formula (V) | 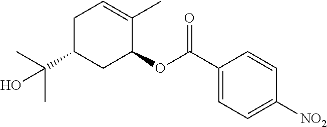 [SMILES: CC1=CC[C@@H](C(C)(C)O)C[C@@H]1OC(=O)c1ccc([N+](=O)[O-])cc1] |
| Formula (VI) | 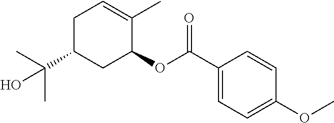 [SMILES: COc1ccc(C(=O)O[C@H]2C[C@H](C(C)(C)O)CC=C2C)cc1] |
| Formula (VII) | 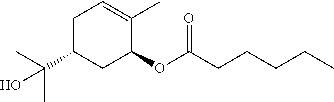 [SMILES: CCCCCC(=O)O[C@H]1C[C@H](C(C)(C)O)CC=C1C] |
| Formula (VIII) | 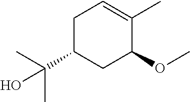 |
| Formula (IX) | 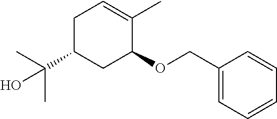 [SMILES: CC1=CC[C@@H](C(C)(C)O)C[C@@H]1OCc1ccccc1] |
| Formula (X) | 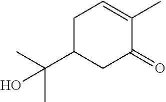 |
| Formula (XI) | 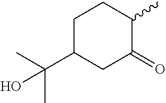 |
| Formula (XII) | 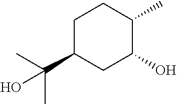 |
| Formula (XIII) | 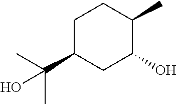 |
| Formula (XIV) | 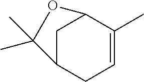 |
| Formula (XV) | 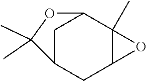 |

and wherein the sustained-release pellet comprises:
a second immediate-release pellet; and
a first sustained-release layer coated on the second immediate-release pellet, which is composed of a first sustained-release composition,
wherein the second immediate-release pellet is composed of a second medicated mixture, and the second medicated mixture comprises:
a second compound, or its geometric isomer, enantiomer, diastereomer or pharmaceutically acceptable salt; and
a second excipient composition,
wherein a content of the second compound in the second medicated mixture is 5-85 wt %, and the second compound comprises sobrerol having the Formula (I) shown above or a sobrerol derivative having a formula selected from the group consisting of Formula (II)-Formula (XV) shown above, and
wherein the first sustained-release composition comprises:
a first water-insoluble polymer; and
a first water-soluble binder, and
wherein the enteric-release pellet comprises:
a third immediate-release pellet; and
a first enteric-release layer coated on the third immediate-release pellet, which is composed of a first enteric-release composition,
wherein the third immediate-release pellet is composed of a third medicated mixture, and the third medicated mixture comprises:
a third compound, or its geometric isomer, enantiomer, diastereomer or pharmaceutically acceptable salt; and
a third excipient composition,
wherein a content of the third compound in the third medicated mixture is 5-85 wt %, and the third compound comprises sobrerol having the Formula (I) shown above or a sobrerol derivative having a formula selected from the group consisting of Formula (II)-Formula (XV) shown above,
wherein the first enteric-release composition comprises:
a first enteric-release substance with pH-dependent solubility; and
a first anti-adherent plasticizer, and
wherein the enteric coated sustained-release pellet comprises:
a fourth immediate-release pellet;
a second sustained-release layer coated on the fourth immediate-release pellet, which is composed of a second sustained-release composition; and
a second enteric-release layer coated on the second sustained-release layer, which is composed of a second enteric-release composition,
wherein the fourth immediate-release pellet is composed of a fourth medicated mixture, and the fourth medicated mixture comprises:
a fourth compound, or its geometric isomer, enantiomer, diastereomer or pharmaceutically acceptable salt; and
a fourth excipient composition,
wherein a content of the fourth compound in the fourth medicated mixture is 5-85 wt %, and the fourth compound comprises sobrerol having the Formula (I) shown above or a sobrerol derivative having a formula selected from the group consisting of Formula (II)-Formula (XV) shown above, and
wherein the second sustained-release composition comprises:
a second water-insoluble polymer; and
a second water-soluble binder, and
wherein the second enteric-release composition comprises:
a second enteric-release substance with pH-dependent solubility; and
a second anti-adherent plasticizer.

2. The multi-particulate pharmaceutical composition as claimed in claim 1, wherein the mixture pellets consist of the first immediate-release pellet and the sustained-release pellet, and a weight ratio of the first compound to the second compound is 1:0.10-10.00.

3. The multi-particulate pharmaceutical composition as claimed in claim 1, wherein the mixture pellets consist of the first immediate-release pellet and the enteric-release pellet, and a weight ratio of the first compound to the third compound is 1:0.10-10.00.

4. The multi-particulate pharmaceutical composition as claimed in claim 1, wherein the mixture pellets consist of the sustained-release pellet and the enteric-release pellet, and a weight ratio of the second compound to the third compound is 1:0.10-10.00.

5. The multi-particulate pharmaceutical composition as claimed in claim 1, wherein the mixture pellets consist of the first immediate-release pellet, the sustained-release pellet and the enteric-release pellet, and a weight ratio of the first compound, the second compound and the third compound is 1:0.10-10.00:0.10-10.00.

6. The multi-particulate pharmaceutical composition as claimed in claim 1, wherein a weight ratio of the first compound to the first excipient composition, a weight ratio of the second compound to the second excipient composition, a weight ratio of the third compound to the third excipient composition, and a weight ratio of the fourth compound to the fourth excipient composition independently are 1:0.3-5.0.

7. The multi-particulate pharmaceutical composition as claimed in claim 1, wherein the first excipient composition, the second excipient composition, the third excipient composition and the fourth excipient composition independently comprise a filler, and the filler comprises at least one of the following ingredients:
microcrystalline cellulose, lactose, starch, corn starch, mannitol, calcium hydrogen phosphate and dicalcium phosphate.

8. The multi-particulate pharmaceutical composition as claimed in claim 1, wherein a weight ratio of the second immediate-release pellet to the first sustained-release composition is 1:0.01-0.2.

9. The multi-particulate pharmaceutical composition as claimed in claim 1, wherein a weight ratio of the second immediate-release pellet, the first water-insoluble polymer and the first water-soluble binder is 1:0.040-0.1000:0.009-0.030.

10. The multi-particulate pharmaceutical composition as claimed in claim 1, wherein the first water-insoluble polymer comprises at least one of the following ingredients:
ethyl cellulose, ethyl acrylate-methyl methacrylate-trimethyl ammonium chloride ethyl methacrylate copolymer, methyl methacrylate-ethyl acrylate copolymer, hydrogenated castor oil, hydrogenated coconut oil, stearic acid and stearyl alcohol, and
wherein the first water-soluble binder comprises at least one of the following ingredients:
hydroxypropyl methyl cellulose, hydroxypropyl cellulose, polyvinylpyrrolidone and polyvinyl alcohol.

11. The multi-particulate pharmaceutical composition as claimed in claim 1, wherein a weight ratio of the third immediate-release pellet to the first enteric-release composition is 1:0.1-0.05.

12. The multi-particulate pharmaceutical composition as claimed in claim 1, wherein a weight ratio of the third immediate-release pellet, the first enteric-release substance with pH-dependent solubility and the first anti-adherent plasticizer is 1:0.01-1:0.005-0.5.

13. The multi-particulate pharmaceutical composition as claimed in claim 1, wherein the first enteric-release substance with pH-dependent solubility comprises at least one of the following ingredients:
an enteric acrylic copolymer, an enteric cellulose derivative, an enteric starch derivative and saturated fatty acid, and
wherein an ingredient of the first anti-adherent plasticizer comprises at least one of the following:
glyceryl monostearate, polysorbate 80 and triethyl citrate(triethyl citrate).

14. The multi-particulate pharmaceutical composition as claimed in claim 1, wherein the sustained-release pellet further comprises a coating layer coated on the sustained-release layer, which is composed of a coating composition.

15. The multi-particulate pharmaceutical composition as claimed in claim 1, wherein the enteric-release pellet further comprises a coating layer coated on the enteric-release layer, which is composed of a coating composition.

16. The multi-particulate pharmaceutical composition as claimed in claim 1, further comprising at least one pharmaceutically acceptable excipient.

17. The multi-particulate pharmaceutical composition as claimed in claim 1, further comprising a capsule to encapsulate the mixture pellets.

18. An immediate-release pellet, consisting of a medicated mixture, wherein the medicated mixture comprises:
a compound, or its geometric isomer, enantiomer, diastereomer or pharmaceutically acceptable salt; and
an excipient composition, which comprises a filler,
wherein a content of the compound in the medicated mixture is 5-85 wt %, and the compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 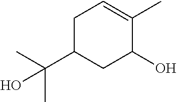 |
| Formula (II) | 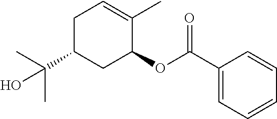 [SMILES: CC1=CC[C@@H](C(C)(C)O)C[C@@H]1OC(=O)c1ccccc1] |
| Formula (III) | 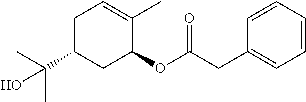 [SMILES: CC1=CC[C@@H](C(C)(C)O)C[C@@H]1OC(=O)Cc1ccccc1] |
| Formula (IV) | 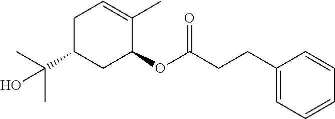 [SMILES: CC1=CC[C@@H](C(C)(C)O)C[C@@H]1OC(=O)CCc1ccccc1] |
| Formula (V) | 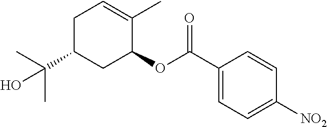 [SMILES: CC1=CC[C@@H](C(C)(C)O)C[C@@H]1OC(=O)c1ccc([N+](=O)[O-])cc1] |
| Formula (VI) | 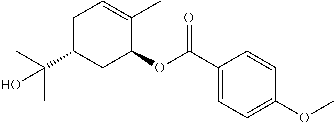 [SMILES: COc1ccc(C(=O)O[C@H]2C[C@H](C(C)(C)O)CC=C2C)cc1] |
| Formula (VII) | 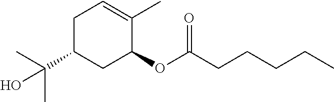 [SMILES: CCCCCC(=O)O[C@H]1C[C@H](C(C)(C)O)CC=C1C] |
| Formula (VIII) | 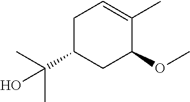 |
| Formula (IX) | 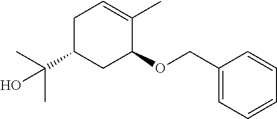 [SMILES: CC1=CC[C@@H](C(C)(C)O)C[C@@H]1OCc1ccccc1] |
| Formula (X) | 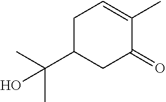 |
| Formula (XI) | 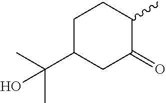 |
| Formula (XII) | 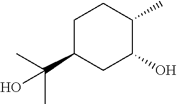 |
| Formula (XIII) | 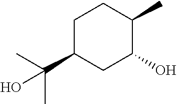 |
| Formula (XIV) | 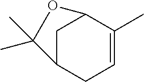 |
| Formula (XV) | 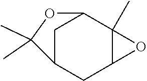 , |

and wherein a weight ratio of the compound to the excipient composition is 1:0.3-5.0, a weight ratio of the compound to the filler is 1:0.15-5.00, and a particle size of the immediate-release pellet is 360-1100 μm.

19. A sustained-release pellet, comprising:
an immediate-release pellet; and
a sustained-release composition coated on the immediate-release pellet to constitute a sustained-release layer,
wherein the immediate-release pellet is composed of a medicated mixture, and the medicated mixture comprises:
a compound, or its geometric isomer, enantiomer, diastereomer or pharmaceutically acceptable salt; and
an excipient composition,
wherein the compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 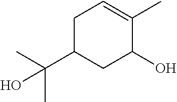 |
| Formula (II) | 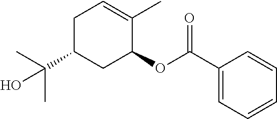 [SMILES: CC1=CC[C@@H](C(C)(C)O)C[C@@H]1OC(=O)c1ccccc1] |
| Formula (III) | 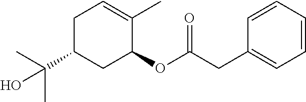 [SMILES: CC1=CC[C@@H](C(C)(C)O)C[C@@H]1OC(=O)Cc1ccccc1] |
| Formula (IV) | 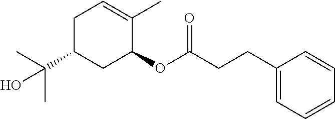 [SMILES: CC1=CC[C@@H](C(C)(C)O)C[C@@H]1OC(=O)CCc1ccccc1] |
| Formula (V) | 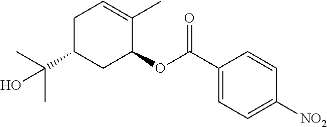 [SMILES: CC1=CC[C@@H](C(C)(C)O)C[C@@H]1OC(=O)c1ccc([N+](=O)[O-])cc1] |
| Formula (VI) | 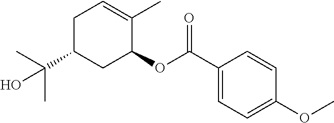 [SMILES: COc1ccc(C(=O)O[C@H]2C[C@H](C(C)(C)O)CC=C2C)cc1] |
| Formula (VII) | 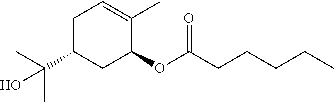 [SMILES: CCCCCC(=O)O[C@H]1C[C@H](C(C)(C)O)CC=C1C] |
| Formula (VIII) | 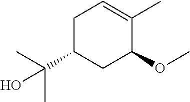 |
| Formula (IX) | 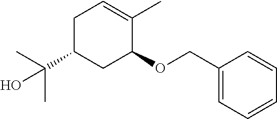 [SMILES: CC1=CC[C@@H](C(C)(C)O)C[C@@H]1OCc1ccccc1] |
| Formula (X) | 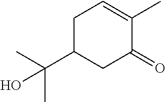 |
| Formula (XI) | 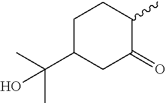 |
| Formula (XII) | 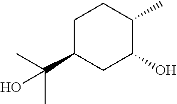 |
| Formula (XIII) | 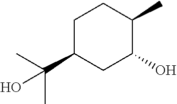 |
| Formula (XIV) | 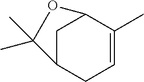 |
| Formula (XV) | 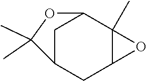 , |

and wherein the sustained-release composition comprises:
a water-insoluble polymer; and
a water-soluble binder, and
wherein:
a content of the compound in the medicated mixture is 5-85 wt %,
a weight ratio of the compound to the excipient composition is 1:0.4-5.5, and a weight ratio of the compound to the filler is 1:0.15-5.00,
a weight ratio of the immediate-release pellet to the sustained-release composition is 1:0.08-0.60, and a weight ratio of the second immediate-release pellet, the water-insoluble polymer and the water-soluble binder is 1:0.040-0.100:0.009-0.030, and
a particle size of the sustained-release pellet is 425-1150 μm.

20. An enteric-release pellet, comprising:
an immediate-release pellet; and
an enteric-release composition coated on the immediate-release pellet to constitute an enteric-release layer,
wherein the immediate-release pellet is composed of a medicated mixture, and the medicated mixture comprises:
a compound, or its geometric isomer, enantiomer, diastereomer or pharmaceutically acceptable salt; and
an excipient composition,
wherein the compound comprises sobrerol having the Formula (I) shown in the following or a sobrerol derivative having a formula selected from the group consisting of Formula (II)-Formula (XV) shown in the following:

| Formula | Compound |
|---|---|
| Formula (I) | 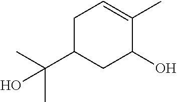 |
| Formula (II) | 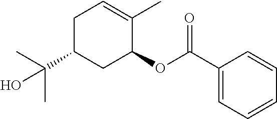 [SMILES: CC1=CC[C@@H](C(C)(C)O)C[C@@H]1OC(=O)c1ccccc1] |
| Formula (III) | 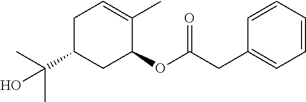 [SMILES: CC1=CC[C@@H](C(C)(C)O)C[C@@H]1OC(=O)Cc1ccccc1] |
| Formula (IV) | 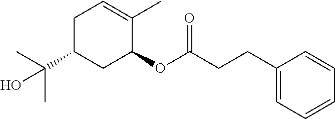 [SMILES: CC1=CC[C@@H](C(C)(C)O)C[C@@H]1OC(=O)CCc1ccccc1] |
| Formula (V) | 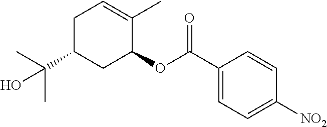 [SMILES: CC1=CC[C@@H](C(C)(C)O)C[C@@H]1OC(=O)c1ccc([N+](=O)[O-])cc1] |
| Formula (VI) | 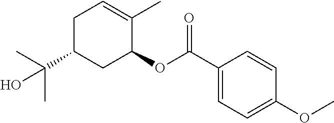 [SMILES: COc1ccc(C(=O)O[C@H]2C[C@H](C(C)(C)O)CC=C2C)cc1] |
| Formula (VII) | 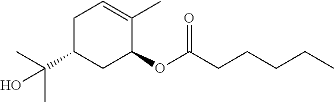 [SMILES: CCCCCC(=O)O[C@H]1C[C@H](C(C)(C)O)CC=C1C] |
| Formula (VIII) | 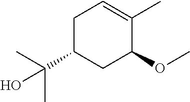 |
| Formula (IX) | 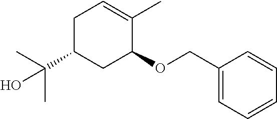 [SMILES: CC1=CC[C@@H](C(C)(C)O)C[C@@H]1OCc1ccccc1] |
| Formula (X) | 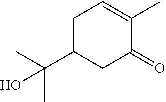 |
| Formula (XI) | 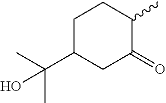 |
| Formula (XII) | 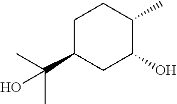 |
| Formula (XIII) | 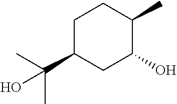 |
| Formula (XIV) | 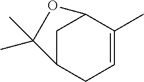 |
| Formula (XV) | 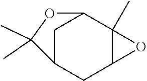 , |

and wherein the enteric-release composition comprises:
an enteric-release substance with pH-dependent solubility, and
an anti-adherent plasticizer, and
wherein:
a content of the compound in the medicated mixture is 5-85 wt %,
a weight ratio of the compound to the excipient composition is 1:0.65-6.00, and a weight ratio of the compound to the filler is 1:0.15-5.00,
a weight ratio of the immediate-release pellet to the enteric-release composition is 1:0.20-0.40, and a weight ratio of the immediate-release pellet, the enteric-release substance with pH-dependent solubility and the anti-adherent plasticizer is 1:0.01-1:0.005-0.5, and
a particle size of the enteric-release pellet is 465-1380 μm.
